(19) United States
(12) Patent Application Publication
(10) Pub. No.: US 2026/0308498 A1
(43) Pub. Date: Oct. 8, 2026

(54) LYMPHOID TISSUES WITH SWITCHABLE PROTEIN GRADIENTS

(71) Applicants: Manuel Quinones PEREZ, Atlanta, GA (US); Ankur SINGH, Marietta, GA (US); Zhe ZHONG, Atlanta, GA (US); GEORGIA TECH RESEARCH CORPORATION, Atlanta, GA (US)

(72) Inventors: Manuel Quinones PEREZ, Atlanta, GA (US); Ankur SINGH, Marietta, GA (US); Zhe ZHONG, Atlanta, GA (US)

(21) Appl. No.: 19/482,476

(22) PCT Filed: May 9, 2024
(86) PCT No.: PCT/US2024/028605

§ 371 Date: Nov. 7, 2025

Related U.S. Application Data
(60) Provisional application No. 63/465,119, filed on May 9, 2023.
(60) Provisional application No. 63/571,731, filed on Mar. 29, 2024.

(51) Int. Cl. C12M 3/00; C07K 16/00; C12M 1/12; C12M 3/06; C12N 5/0781; C12N 5/0783; C12N 5/0784

(52) U.S. Cl. CPC C12M 21/08; C07K 16/00; C12M 23/16; C12M 25/14; C12N 5/0635; C12N 5/0636; C12N 5/0639; C12N 2503/02

(57) ABSTRACT

An organ-on-chip system having a gradient of a stimulating agent (e.g., chemokine) and a plurality of immune cells expressing different phenotypes relative to their placement within the gradient and methods of using the same.

### CROSS REFERENCE TO RELATED APPLICATIONS

This application claims the benefit of priority to U.S. Provisional Application No. 63/465,119, filed May 9, 2023, and U.S. Provisional Application No. 63/571,731, filed Mar. 29, 2024, which are each incorporated by reference herein in their entireties.

### BACKGROUND

The process of antibody formation and decision-making against infection and vaccination takes place in lymph nodes and similar lymphoid tissues and relies on the spatial organization of immune cells, which then regulates the temporal fate of the immune cells upon external stimulus. This spatial organization happens in the body through the secretion of chemokine proteins by stromal cells. The state-of-the-art immune tissues, either as 2D aggregates of cells or as 3D hydrogels, lack the ability to control the spatial and temporal differentiation of immune cells in response to external stimuli. There exists a need for immunocompetent lymphoid tissue systems.

### SUMMARY

Disclosed herein is a lymphoid tissue system with switchable protein gradients. In one embodiment, serpentine microchannels can be created to develop the gradient of immune cell attracting chemokine across the hydrogels that encapsulate immune cells. The exemplary system can allow for simple crosslinking of immune tissues on microfluidic devices through a hole in the middle of the device fabricated without the need for extensive engineering training. Due to the switchable nature of the chemokine gradient, the exemplary system can allow for unexpected polarization of immune cell phenotypes, which can impact the differentiation fate of immune cells.

In an aspect, provided is an organ-on-chip system, including: a fluidic substrate (e.g., microfluidic) having a plurality of channels and a chamber, the chamber being configured to house a hydrogel embedded with a plurality of immune cells or placed thereon; wherein the plurality of channels can include at least one channel connected to the chamber to deliver a stimulating agent to the plurality of immune cells of the hydrogel; wherein the hydrogel can include a gradient of the stimulating agent (e.g., wherein each of the plurality of channels closer to the first inlet can have a lower concentration of the stimulating agent and each of the plurality of channels closer to the second inlet can have a higher concentration of the stimulating agent); and wherein different regions of the hydrogel each with different portions of the plurality of immune cells can express different phenotypes relative to their placement within the gradient.

In another aspect, provided is a method of stimulating a plurality of immune cells, the method comprising culturing a plurality of immune cells on any one of the disclosed organ-on-chip systems.

In yet another aspect, provided is a cell produced by any of the disclosed methods of stimulating a plurality of immune cells.

In yet another aspect, provided is a method of monitoring disease progression, the method including: a) culturing a plurality of immune cells on any of the disclosed organ-on-chip systems; and b) observing the plurality of immune cells over a period of time (e.g., hours, days, weeks, months); wherein the plurality of immune cells are diseased or abnormal.

In yet another aspect, provided is a method of determining a response to a therapeutic intervention, the method including: a) culturing a plurality of immune cells on any of the disclosed organ-on-chip systems; and b) subjecting the plurality of immune cells to the therapeutic intervention.

In yet another aspect, provided is a method of generating antibodies, the method including: a) culturing a plurality of immune cells on any one of the disclosed organ-on-chip systems; and b) collecting antibodies produced by the plurality of immune cells.

In yet another aspect, provided is a method of generating antibodies for 24 days or longer, the method including: a) culturing a plurality of peripheral blood mononuclear cells (PBMCs) on any of the disclosed organ-on-chip systems; and b) collecting antibodies produced by the plurality of PBMCs.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1J depict lymphocyte-encapsulated tissues with switchable gradients in organ-on-chip. FIGS. 1A-1C show schematics representing device design (FIG. 1A), the brightfield images of PDMS devices with embedded PEG-4MAL hydrogels organoid (FIG. 1B), and fluorescent images showing the formation of FITC-BSA gradient in the device (FIG. 1C). FIG. 1D shows analysis of FITC-BSA gradient development within the hydrogel 12 hours post-initiation of the study as function of hydrogel diameter. FIGS. 1E-1F show controlled polarization of light zone and dark zone B cells in microfluidic devices under CXCL12 chemokine gradient inside and vertical directions, in tonsil organoids (FIG. 1E) and PBMC organoids (FIG. 1F). Images are representative of 3 devices from a single donor of tonsil or PBMCs. FIG. 1G shows the CXCR4 proportion analysis based on area polarization in immunofluorescence staining of CXCR4 and CD83 with 3 PBMC donors in technical duplicates of PBMC-organoid devices. FIG. 1H shows secreted IgG1 in PBMC-organoid devices with chemokine gradient, chemokine, and control. Mean±S.D. Brown-Forsythe and Welch one-way ANOVA with Dunnett's T3 multiple comparisons test. N=4, each dot represents a PBMC-organoid devices technical replicate from a particular donor. FIG. 1I shows secreted cytokines in PBMC-organoid devices with chemokine gradient, chemokine, and control (no gradient). Individual devices are indicated. Mean±S.D. Brown-Forsythe and Welch one-way ANOVA with Dunnett's T3 multiple comparisons test. N=4, each dot represents a PBMC-organoid devices technical replicate from a particular donor. FIG. 1J shows a confocal microscopy-based 3D projection of CD4+ T cells, CXCR4+ cells, and DAPI in PBMC-organoid devices in microfluidic devices under CXCL12 chemokine gradient and no chemokine control. Images are representative of three independent donors.

FIGS. 2A-2K depict bioengineered tonsil-derived hydrogel-based lymphoid organoids. FIG. 2A shows imaging mass cytometry (IMC) of human tonsil tissues demonstrating cellular and extracellular microenvironment with Collagen type I, CD4, KI67, CD20, CD21. Images are representative of results from 3 donors. FIG. 2B shows IMC of human tonsil tissues demonstrating localization of FDCs (CD21+), B cells (CD20+), T cells (CD4+), and Follicular Helper T cells (CD4+PD1+). Images are representative of results from 3 donors.

FIG. 2C shows workflow for tonsil tissue disruption and organoids culture preparation. FIG. 2D shows fold changes in live tonsil B cells (CD20+) in tonsil organoids cultured with soluble CD40L or cellular membrane-bound CD40L (CD40L-T cell mimic). N=4, each dot represents an organoid. One-way ANOVA with Tukey's posthoc test. FIG. 2E shows the percentage of CD20+ B cells in tonsil organoids cultured with different cell densities (0, 20000, 40000) of HK-FDCs, incubated with Hemagglutinin (HA) antigen. N=3, each dot represents an organoid. One-way ANOVA with Tukey's posthoc test. FIG. 2F shows mechanical properties of PEG-4MAL hydrogels at different weight percentages with two crosslinking densities (4:1:1.5, 4:1:0.75, where ratio represents PEG-4MAL: Adhesive peptide: Crosslinker). N=5, each dot represents an organoid. Two-way ANOVA with Tukey's posthoc test. FIG. 2G shows representative flow cytometry gating of B cell major phenotypes: Naïve B cells, Activated B cells, Germinal center B cells, and Plasmablasts, and the count of tonsil GC B cells differentiated in 4 different weight percentages of PEG-4MAL hydrogel with two crosslinking densities after 4 days. N=4, each dot represents an organoid. Two-way ANOVA with Tukey's posthoc test. FIG. 2H shows cell distribution in high and low cross-linking density after 4 days.

FIG. 2I shows representative flow cytometry gating of CD19 and Annexin V for tonsil apoptotic CD19+ B cells for 10 kDa and 20 kDa PEG-4MAL, and 20 kDa PEG-4VS. Percentage of Annexin V+ CD19+ apoptotic B cells after 4 days. N=5, each dot represents an organoid. One-way ANOVA with Tukey's posthoc test. FIG. 2J shows a count of tonsil GC B cells differentiated in PEG-4MAL vs. PEG-4VS. N=5, each dot represents an organoid. One-way ANOVA with Tukey's posthoc test. FIG. 2K shows representative flow cytometry gating of tonsil B cell phenotypes in PEG-4MAL organoids with GFO+RGD peptides and the count of GC B cells after 4 days with different peptides combination. N=3, each dot represents an organoid. Two-way ANOVA with Tukey's posthoc test.

FIGS. 3A-3P depict that PBMC-derived hydrogel-based lymphoid organoids induce robust and sustainable B cell differentiation. FIG. 3A shows a histogram and the fluorescent intensity of CD40L expression for organoids with RGD+GFO vs. RDG+GFO peptides. Mean±S.D. Two-way ANOVA with Tukey's multiple comparisons test. N=4, each dot represents an organoid technical replicate from a donor. FIG. 3B shows representative flow cytometry of B cell phenotypes in tonsil organoids and counts of GC B cells on D4 and D12 in response to antigen (inactivated H1N1 virus) stimulation. Mean±S.D. Two-way ANOVA with Tukey's multiple comparisons test. N=5, each dot represents an organoid technical replicate from a donor. FIG. 3C shows immunofluorescence staining with AID protein in tonsil tissues and organoids on D12. Data representative of 3 tonsil donors. FIG. 3D shows counts of GC B cells over 24-day tonsil organoids culture. Mean±S.D. Brown-Forsythe and Welch one-way ANOVA with Dunnett's T3 multiple comparisons test. N=5, each dot represents an organoid technical replicate. FIG. 3E shows immunofluorescence staining comparing tonsil organoids and PBMC organoids on D12. Data are representative of 3 separate organoid technical replicates from a donor. FIG. 3F shows immunofluorescence staining of inactivated H1N1 virus antigen (pink) colocalization with human FDCs (white) and pre-stained PBMC cells (green).

FIG. 3G shows representative flow cytometry of B cell phenotypes in PBMC-derived hydrogel-based organoids and the count of GC B cells on D4 and D12 in response to antigen (inactivated H1N1 virus) stimulation. Mean±S.D. Two-way ANOVA with Tukey's multiple comparisons test. N=3, each dot represents an organoid technical replicate from a donor. FIG. 3H shows a count of ASC cells on D4 and D12 in response to antigen (inactivated H1N1 virus) stimulation in PBMC-derived hydrogel-based organoids. Mean±S.D. Two-way ANOVA with Tukey's multiple comparisons test. N=3, each dot represents an organoid technical replicate from a donor. FIG. 3I shows a count of ASC cells on D4 and D12 in response to antigen (inactivated H1N1 virus) stimulation in PBMC-derived 2D coculture with CD40L-TCM. Mean±S.D. Two-way ANOVA with Tukey's multiple comparisons test. N=3, each dot represents an organoid technical replicate from a donor. FIGS. 3J-3L show counts of GC B cells (FIG. 3J), plasma cells (FIG. 3K), and memory B cells (FIG. 3L) over 24-day PEG-4MAL-based PBMC organoid cultures. Mean±S.D. Brown-Forsythe and Welch one-way ANOVA with Dunnett's T3 multiple comparisons test. N=5, each dot represents an organoid technical replicate. FIG. 3M shows a count of follicular helper T cells on D4 and D16 in tonsil-derived and PBMC-derived hydrogel-based organoids. Mean±S.D. Two-way ANOVA with Tukey's multiple comparisons test. N=3, each dot represents an organoid technical replicate from a donor. FIG. 3N shows immunofluorescence staining capturing the interaction of B cell (pink), CD4 T cell (green), and follicular helper T cell (green, white). FIG. 3O shows bulk RNA-seq KEGG pathway analysis of CD19+ B cells from a pool of 3 tonsil donors and 3 PBMC donors on DO. FIG. 3P shows a count of plasma cells on D12 and D24 in tonsil-derived and PBMC-derived hydrogel-based organoids with purified naïve B cells as starting population. Mean±S.D. Two-way ANOVA with Tukey's multiple comparisons test. N=3, each dot represents an organoid technical replicate from a donor.

FIGS. 4A-4G depict that PBMC-derived hydrogel-based organoids capture donor variability and adjuvant response. FIG. 4A shows fold change of GC B cells, ASCs, and plasma cells over 24 days of PBMC organoids culture, from 3 donors, when challenged with antigen (inactivated H1N1 virus) and antigen with TLR7/8 agonist R848. Mean±S.D. Two-way ANOVA with Sidak's multiple comparisons test. N=3, each dot represents an organoid technical replicate from a donor. FIG. 4B shows brightfield images of H1N1-specific ELISPOT and quantification of spots for PBMC-derived organoids and tonsil-derived organoids on D12 and D24 with control, inactivated H1N1 virus alone, and inactivated H1N1 with R848. Mean±S.D. Ordinary one-way ANOVA with Tukey's multiple comparisons test. N=3, each dot represents an organoid technical replicate from the same PBMC or tonsil donor. FIG. 4C shows H1N1-specific ELISPOT of spots for additional 5 PBMC donors-derived organoids on D24, treated with inactivated H1N1 virus alone and inactivated H1N1 with R848. Mean±S.D. Unpaired, two-tailed t-test with Welch correction as appropriate. N=3, each dot represents an organoid technical replicate from the same PBMC donor. FIG. 4D shows IgG response to H3/Hongkong flu or H1/Michigan flu in PBMC-derived organoid supernatant from two PBMC donors. N=3, each dot represents an organoid technical replicate from a donor. Unpaired, two-tailed t-test with Welch's correction and accounted for heteroscedasticity. FIG. 4E shows IgG response to H1/Michigan flu in PBMC-derived organoid supernatant from five additional PBMC donors. Each dot represents a donor (an average of four technical organoid replicates of that donor). Unpaired, two-tailed t-test with Welch's correction and accounted for heteroscedasticity. FIG. 4F shows representative flow cytometry gating and quantification of H1N1-specific total CD19+ B cells, GC B cells, and ASCs for H1N1 alone, R848 alone, and H1N1+R848. Mean±S.D. Brown-Forsythe and Welch one-way ANOVA with Dunnett's T3 multiple comparisons test and accounted for heteroscedasticity. N=3, each dot represents an organoid technical replicate. FIG. 4G shows fabrication of organoids with purified naïve B cells from PBMCs, and the count of GC B cells, ASCs, IgG+GC B cells, and IgM+GC B cell on D12 with H1N1, R848, and H1N1+R848. Mean±S.D. Ordinary one-way ANOVA with Tukey's multiple comparisons test and accounted for heteroscedasticity. N=3, each dot represents an organoid technical replicate from the same donor.

FIGS. 5A-5E depict ex vivo lymphoid organoids form dark zone and light zone compartments of GCs. FIG. 5A shows differential gene expression of organoid sorted CD19+ B cells and CD138+ B cells from tonsil and PBMC-derived organoids, exposed to H1N1+R848. FIGS. 5B-5C show flow cytometry gating for dark zone (DZ: CXCR4+CD83−) and light zone (LZ: CXCR4-CD83+) GC B cells. The count of DZ and LZ GC B cells over 24-day tonsil organoid culture (5 donors) or PBMC organoid culture (4 donors), exposed to inactivated H1N1 virus antigen alone (FIG. 5B) or H1N1+R848 (FIG. 5C). Antigen used was inactivated H1N1 virus. N=3, each dot represents an average of 3 organoid technical replicates from a donor. FIG. 5D shows CXCR4 and CD83 immunofluorescence staining of PBMC organoids culture, when incubated with H1N1 alone and with H1N1+R848. Images representative of 3 organoid technical replicates from a donor. FIG. 5E shows fold change in the number of CD83+ clusters based on immunofluorescence staining analysis of five independent PBMC donors. N=3, each dot represents an average of 3 organoid technical replicates from a donor.

FIGS. 6A-6N depict bioengineered tonsil-derived hydrogel-based lymphoid organoids. FIG. 6A shows Cyclic Immunofluorescence of a human tonsil tissue stained with CD4, CD20, CD21, Collagen type I, VCAM1, and Ki67. Images are representative of results from 3 donors. FIG. 6B shows Atomic Force Microscopy (AFM)-based stiffness measurement of human tonsil tissues from 3 donors. Each dot for a donor represents an AFM measurement at a different spot in tissue. Mean±S.D. FIG. 6C shows mechanical properties of PEG-4MAL hydrogels at different weight percentages with fixed crosslinker densities. G′ represents storage modulus, G″ represents loss modulus. Mean±S.D. Brown-Forsythe and Welch one-way ANOVA with Dunnett's T3 multiple comparisons test. N=5, each dot represents an organoid technical replicate. FIG. 6D shows a workflow for tonsil organoids culture preparation. FIG. 6E shows fold changes in live tonsil B cells (CD20+) in tonsil organoids cultured with soluble CD40L or cellular membrane-bound CD40L (CD40L-T cell mimic). Mean±S.D. Brown-Forsythe and Welch one-way ANOVA with Dunnett's T3 multiple comparisons test. N=4, each dot represents an organoid technical replicate from the same donor. FIG. 6F shows the percentage of CD20+ B cells in tonsil organoids cultured with different cell densities (0, 20000, 40000) of HK-FDCs, incubated with Hemagglutinin (HA) antigen. Mean±S.D. Ordinary one-way ANOVA with Tukey's multiple comparisons test. N=3, each dot represents an organoid technical replicate from the same donor. FIG. 6G shows representative flow cytometry gating of B cell major phenotypes: naïve B cells, activated B cells, germinal center B cells, and ASCs, and the count of tonsil GC B cells differentiated in 4 different weight percentages of PEG-4MAL hydrogel with two crosslinking densities after 4 days (4:1:1.5, 4:1:0.75, where ratio represents PEG-4MAL: Adhesive peptide: Crosslinker and the numbers refer to the molar ratio of Maleimides to Thiols). Mean±S.D. Two-way ANOVA with Tukey's multiple comparisons test. N=4, each dot represents an organoid technical replicate from a donor. FIG. 6H shows cell distribution in PEG-4MAL hydrogels with high and low cross-linking density after 4 days. Images are representative of results from 3 donors. FIG. 6I shows representative flow cytometry gating of CD19 and Annexin V for tonsil apoptotic CD19+ B cells for 10 kDa and 20 kDa PEG-4MAL and 20 kDa PEG-4VS. Percentage of Annexin V+CD19+ apoptotic B cells after 4 days. Mean±S.D. Ordinary one-way ANOVA with Tukey's multiple comparisons test. N=5, each dot represents an organoid technical replicate from the same donor. FIG. 6J shows a count of tonsil GC B cells differentiated in PEG-4MAL vs. PEG-4VS. Mean±S.D. Brown-Forsythe and Welch one-way ANOVA with Dunnett's T3 multiple comparisons test. N=5, each dot represents an organoid technical replicate. FIG. 6K shows representative flow cytometry gating of tonsil B cell phenotypes in PEG-4MAL organoids with GFO+RGD peptides and the count of GC B cells after 4 days with different peptide combinations. Mean±S.D. Two-way ANOVA with Tukey's multiple comparisons test. N=3, each dot represents an organoid technical replicate from a donor. FIG. 6L shows the count of CD19+CD38hiCD27+CD138+ ASCs after 4 days with different peptides combination. Mean±S.D. Two-way ANOVA with Tukey's multiple comparisons test. N=3, each dot represents an organoid technical replicate from a donor. FIG. 6M shows a count of tonsil GC B cells differentiated in PEG-4MAL organoids with GFO+RGD peptides or GFO+RGD+REDV peptides in the presence and absence of α4β1 inhibitor. Mean±S.D. Brown-Forsythe and Welch one-way ANOVA with Dunnett's T3 multiple comparisons test. N=5, each dot represents an organoid technical replicate. FIG. 6N shows a schematic demonstrating the final composition of organoids for further studies.

FIGS. 7A-7K depict transcriptomic, BCR, clonal, somatic hypermutation changes in human lymphoid organoids. FIGS. 7A-7C show bulk RNA-seq pathway pathway analysis of CD138+ B cells from a pool of 3 tonsil donors and 3 PBMC donors on D12 or 24. 10 organoids per donor were formed and 30 organoids, representing the three donors or PBMC or tonsil, were pooled. Bulk RNA-seq was performed on day 12 on organoids exposed to H1N1 alone (FIG. 7A), H1N1 alone in comparison with those exposed to the combination of H1N1 and R848 (FIG. 7B), and on day 24 on organoids exposed to H1N1 alone versus H1N1+R848 (FIG. 7C). FIG. 7D shows single cell RNA sequencing and the UMAP projection of B cell clusters, each distinguished by a unique color, across a 24-day timeline of PBMC-derived organoid culture. The distribution represents combined signature of organoids treated with H1N1 alone and H1N1+R848. N=10 organoids from single donor were treated with H1N1 alone or H1N1+R848. FIG. 7E is a heatmap showing the expression of B cell genes in each cluster. FIG. 7F shows a UMAP projection of B cell clusters with colors annotating the clusters of B cell immunoglobulin (Ig) classes across all clusters in B cells from organoids exposed to Ag and Ag+R848. FIG. 7G is a bar plot demonstrating the percentage of the immunoglobulin classes in FIG. 7F. FIG. 7H shows a UMAP projection demonstrating clone emergence in B cells from organoids exposed to Ag and Ag+R848. FIG. 7I shows B cell clone sizes as a function of organoid culture duration and exposure to Ag and Ag+R848. FIG. 7J is a violin plot representing somatic hypermutation levels as the number of heavy chain (HC) complementarity-determining region (CDR) in B cells from organoids exposed to Ag and Ag+R848. FIG. 7K shows the number of HC CDR mutations as a function of organoid culture duration and exposure to Ag and Ag+R848.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Organ-on-Chip Systems

In an aspect, provided is an organ-on-chip system, including: a fluidic substrate (e.g., microfluidic) having a plurality of channels and a chamber, the chamber being configured to house a hydrogel embedded with a plurality of immune cells or placed thereon; wherein the plurality of channels can include at least one channel connected to the chamber to deliver a stimulating agent to the plurality of immune cells of the hydrogel; wherein the hydrogel can include a gradient of the stimulating agent (e.g., wherein each of the plurality of channels closer to the first inlet can have a lower concentration of the stimulating agent and each of the plurality of channels closer to the second inlet can have a higher concentration of the stimulating agent); and wherein different regions of the hydrogel each with different portions of the plurality of immune cells can express different phenotypes relative to their placement within the gradient.

In some aspects, the hydrogel can include a synthetic polymer. For example, in some aspects, the hydrogel can include polyethylene glycol (PEG). In some such aspects, the hydrogel can include 4-arm PEG-maleimide (PEG-4MAL), 4-arm PEG-acrylate (PEG-4A), 4-arm PEG-vinylsulfone (PEG-4S) 4-arm PEG-norbornene (PEG-4NB), 8-arm PEG-maleimide (PEG-8MAL), 8-arm PEG-norbornene (PEG-8NB), or any combination thereof. In some such aspects, the hydrogel can include 10 kDa PEG-4MAL or 20 kDa PEG-4MAL.

In other aspects, the hydrogel can include a natural polymer. For example, in some aspects, the hydrogel can include collagen, alginate, gelatin, fibronectin, fibrinogen, laminin, hyaluronic acid, extracellular matrix, Matrigel, or any combination thereof.

In yet other aspects, the hydrogel can include a combination of any one or more of the synthetic polymers described above and any one or more of the natural polymers described above.

In some aspects, the hydrogel can be functionalized with one or more peptides. In some such aspects, the one or more peptides can include GYGGGP(GPP)5GFOGER(GPP)5GPC, where O=hydroxyproline, GRGDSPC, GREDVGC, or any combination thereof. In some aspects, the one or more peptides can be cyclic. In some aspects, the hydrogel can be additionally or alternatively functionalized with one or more nucleic acids and/or small molecules.

In some aspects, the stimulating agent can include a chemokine or a cytokine. In some aspects, the chemokine can be CXCL12, CXCL13, or a combination thereof.

In some aspects, the stimulating agent can include a small molecule. In some aspects, the small molecule can be an EZH2 inhibitor, a Bcl6 inhibitor, or a combination thereof. In some aspects, the small molecule can be a TLR7/8 agonist.

In some aspects, the hydrogel can further include two opposing gradients of two different stimulating agents. For example, in some such aspects, the first stimulating agent can be an EZH2 inhibitor, and the second stimulating agent can be a Bcl6 inhibitor. In other such aspects, the first stimulating agent can be a first EZH2 inhibitor, and the second stimulating agent can be a second EZH2 inhibitor. In yet other such aspects, the first stimulating agent can be a first Bcl6 inhibitor, and the second stimulating agent can be a second Bcl6 inhibitor.

In some aspects, the organ-on-chip system can further include a first inlet for a cell culture media and a second inlet for the stimulating agent. In some such aspects, each of the plurality of channels can be in fluid communication with the first inlet and the second inlet; and each of the plurality of channels closer to the first inlet can have a lower concentration of the stimulating agent and each of the plurality of channels closer to the second inlet can have a higher concentration of the stimulating agent.

In some aspects, the plurality of immune cells can include B cells and/or T cells, dendritic cells, and subtypes, monocytes, macrophages, natural killer cells, neutrophils, follicular dendritic cells, and/or stromal cells.

In some aspects, the plurality of immune cells can include B cells. In some such aspects, the B cells can be derived from tonsil tissue. In other such aspects, the B cells can be derived from peripheral blood mononuclear cells (PBMCs). In yet other such aspects, the B cells can be derived from spleen tissue, Peyer's patches, a secondary lymphoid organ, or a tertiary lymphoid organ.

In some aspects, the B cells can be polarized into a light zone and a dark zone.

In some aspects, the plurality of immune cells can include T cells and/or stromal cells.

In some aspects, the plurality of immune cells can be human.

### Methods

In an aspect, provided is a method of stimulating a plurality of immune cells, the method comprising culturing a plurality of immune cells on any one of the disclosed organ-on-chip systems.

In some aspects, the plurality of immune cells can include B cells and/or T cells, dendritic cells, and subtypes, monocytes, macrophages, natural killer cells, neutrophils, follicular dendritic cells, and/or stromal cells.

In some aspects, the method can further include collecting the stimulated immune cells and using said stimulated immune cells for immunotherapy.

In another aspect, provided is a cell produced by any of the disclosed methods of stimulating a plurality of immune cells.

In some aspects, the cell can be a B cell, T cell, dendritic cell, and subtypes monocyte, macrophage, natural killer cell, neutrophil, follicular dendritic cell, or stromal cell. In some aspects, the cell can be a B cell or a T cell (e.g., CAR T cell).

In some aspects, the cell can be human.

In another aspect, provided is a method of monitoring disease progression, the method including: a) culturing a plurality of immune cells on any of the disclosed organ-on-chip systems; and b) observing the plurality of immune cells over a period of time (e.g., hours, days, weeks, months); wherein the plurality of immune cells are diseased or abnormal.

In some aspects, the plurality of immune cells can be cancerous, infected, or defective (e.g., for autoimmune diseases). In some such aspects, the plurality of immune cells can include lymphoma cells or multiple myeloma cells. In other such aspects, the plurality of immune cells can be infected with tuberculosis, RSV, HIV, malaria, coronavirus, or influenza. In yet other such aspects, the plurality of immune cells can be consistent with lupus, rheumatoid arthritis, Evan's syndrome, or Hyper X-IgM.

In other aspects, the plurality of immune cells can be healthy, and step a) can further include exposing the plurality of immune cells to a pathogen. In some such aspects, the pathogen can be tuberculosis, RSV, HIV, malaria, coronavirus, or influenza.

In some aspects, step b) can further include identifying live/dead cells, identifying cell markers or phenotypes, and/or monitoring cell growth.

In another aspect, provided is a method of determining a response to a therapeutic intervention, the method including: a) culturing a plurality of immune cells on any of the disclosed organ-on-chip systems; and b) subjecting the plurality of immune cells to the therapeutic intervention.

In some aspects, the plurality of immune cells can be diseased or abnormal. For example, in some aspects, the plurality of immune cells can be cancerous, infected, or defective. In some such aspects, the plurality of immune cells can include lymphoma cells or multiple myeloma cells. In other such aspects, the plurality of immune cells can be infected with tuberculosis, RSV, HIV, malaria, coronavirus, or influenza. In yet other such aspects, the plurality of immune cells are consistent with lupus, rheumatoid arthritis, Evan's syndrome, or Hyper X-IgM.

In some aspects, the therapeutic intervention can be a therapeutic agent, chemotherapy, radiation, high-intensity focused ultrasound, or any combination thereof.

In other aspects, the plurality of immune cells can be healthy. In some such aspects, the therapeutic intervention can be a vaccination. In some such aspects, the vaccination can be directed to tuberculosis, RSV, HIV, malaria, coronavirus, or influenza.

In some aspects, the method can further include: c) observing the plurality of immune cells over a period of time (e.g., hours, days, weeks, months).

In some aspects, step c) can further include identifying live/dead cells, identifying cell markers or phenotypes, and/or monitoring cell growth.

In another aspect, provided is a method of generating antibodies, the method including: a) culturing a plurality of immune cells on any one of the disclosed organ-on-chip systems; and b) collecting antibodies produced by the plurality of immune cells.

In some aspects, step a) can further include B cell clone development.

In some aspects, the antibodies can include monoclonal and/or polyclonal antibodies.

In some aspects, the antibodies can include IgG (e.g., IgG1 and/or IgG2), IgA, IgM, IgD, IgE, or any combination thereof.

In another aspect, provided is a method of generating antibodies for 24 days or longer (e.g., 25 days or longer, 26 days or longer, 27 days or longer, 28 days or longer, 29 days or longer, 30 days or longer, 31 days or longer, 32 days or longer, 33 days or longer, 34 days or longer, 35 days or longer), the method including: a) culturing a plurality of peripheral blood mononuclear cells (PBMCs) on any of the disclosed organ-on-chip systems; and b) collecting antibodies produced by the plurality of PBMCs.

In some aspects, step a) can further include B cell clone development.

In some aspects, the antibodies can include monoclonal antibodies. In some such aspects, the antibodies can include IgG (e.g., IgG1 and/or IgG2), IgA, IgM, IgD, IgE, or any combination thereof.

### Examples

### Example 1: Lymphocyte-Encapsulated Tissues with Switchable Gradients in Organ-On-Chip (LETSGO)

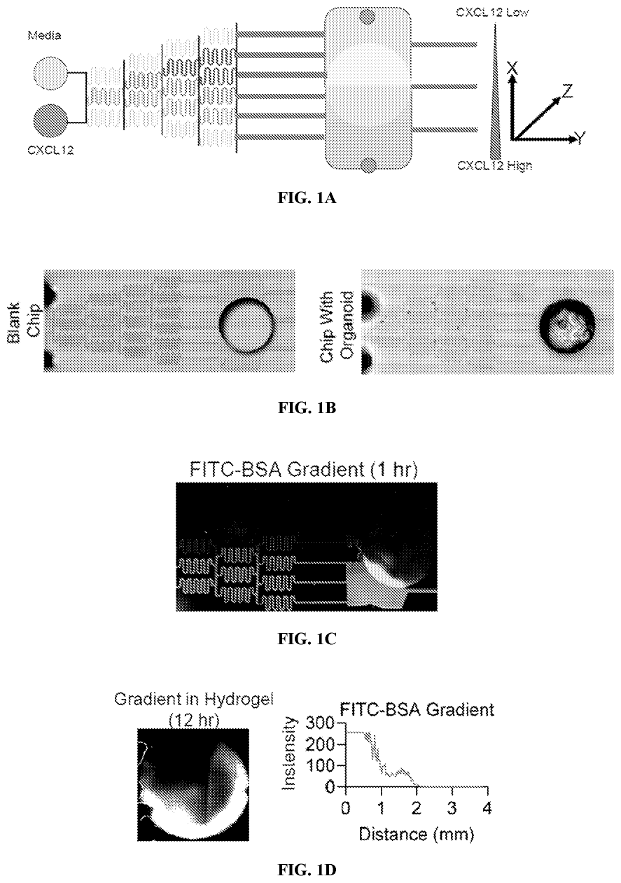

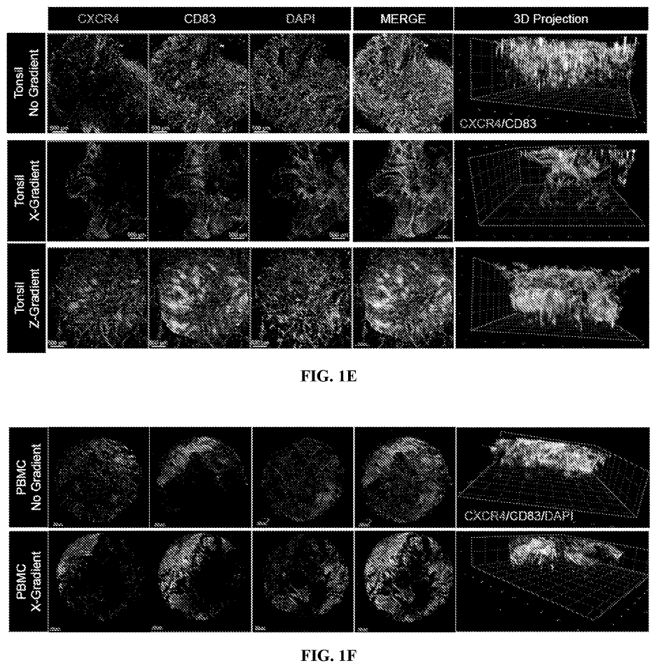

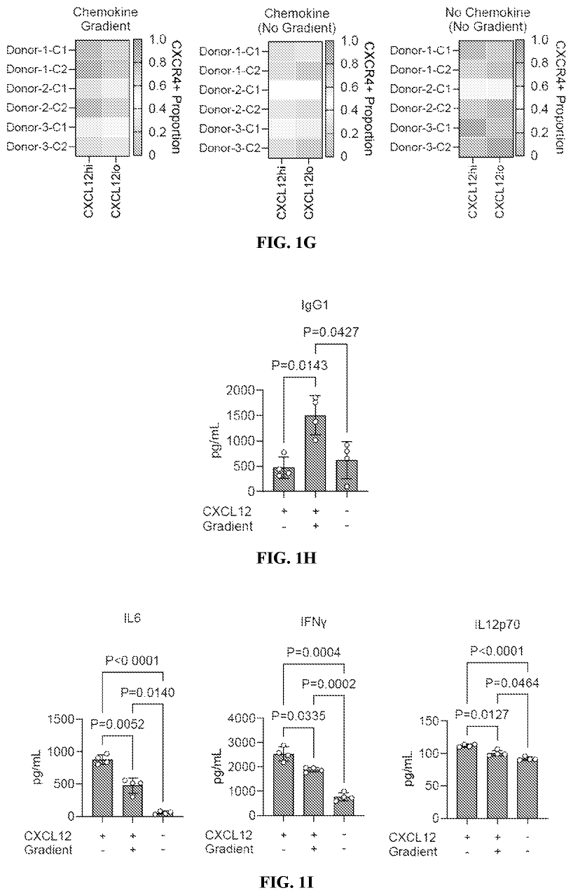

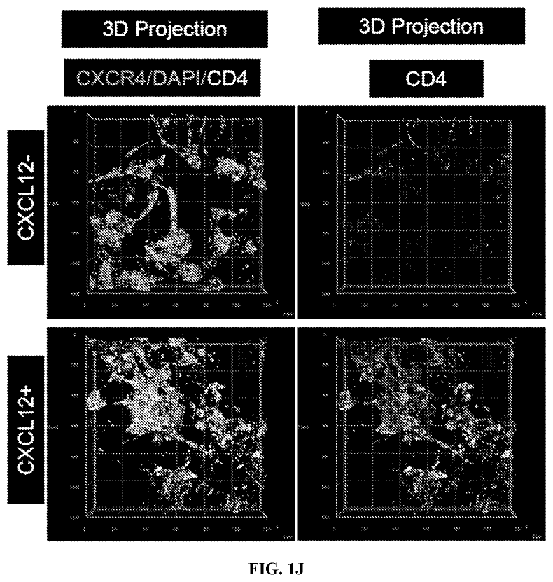

A study was conducted which demonstrated that bulk organoids formulated using PEG-4MAL hydrogels can capture donor heterogeneity, impaired immune response in DLBCL patients, and the conditional differences of antigen and adjuvant exposure, along with recapitulation of selective aspects of the formation of light and dark zone structures. However, these organoids lack the stromal cells that facilitate spatial organization in GCs in vivo through the secretion of chemokines. As described earlier, dark zone/light zone polarization is regulated by the expression of the chemokines CXCL13, which signals through CXCR5, in the light zone, and CXCL12, which signals through CXCR4, in the dark zone. While the HK-FDCs acting as stromal cells in the organoids reportedly secrete low levels of CXCL13 [36], there is no source of CXCL12. The study, therefore, hypothesized that microfluidics-based development of a CXCL12 gradient could provide controlled separation of the light and dark zone structures in lymphoid organoids. To test this hypothesis, the study developed a lymphoid organ-on-chip with lymphocyte-encapsulated hydrogels and CXCL12 gradient (FIG. 1A). Here, a serpentine microchannels was created to establish the gradient of CXCL12 across the PEG-4MAL hydrogel-based B cell organoids. Flowing PEG-4MAL and crosslinker solution in the microfluidic devices is challenging due to rapid crosslink formation; therefore, the hydrogel-based organoids were in situ crosslinked on-chip through a 4 mm diameter hole in the middle of the device (FIG. 1B), fabricated without the need for extensive engineering training. We confirmed protein gradient formation by using FTTC-BSA (FIG. 1C) and observed that the gradient reached across the entire circumference of the side facing high FITC-BSA flow and developed a protein gradient as a function of organoid diameter (FIG. 1D).

To study the effect of the CXCL12 gradient, microfluidic devices with PEG-4MAL gel-based tonsil and PBMC organoids were connected to two inlet lines containing media pumped at 0.5 L/min. In devices treated with CXCL12, one of the syringes was replaced with media containing 100 ng/mL of CXCL12 after 2 days in culture. Devices were placed inverted over a collection dish for media collection for 8 days. We observed dark and light zone GC B cells distributed randomly in the tonsil organoids in conditions that lacked CXCL12. However, when the CXCL12 chemokine gradient was applied across the device in the X-direction, it was observed that CXCR4+ B cells were attracted to the chamber side conforming to the chemokine gradient formed in a sideway (FIG. 1E). Similarly, when the chemokine gradient was applied in the Z-direction, it was observed that CXCR4+ B cells were attracted to the top, conforming to the chemokine gradient formed upside down (FIG. 1E). We next tested whether a similar phenomenon could be observed in PBMC-derived organoids on chip. As indicated in FIG. 1F, PBMCs also polarized into the dark zone and light zone compartments, similar to tonsil organoids.

To quantitatively assess how chemokines affected the spatial organization of CXCR4 and CD83, the study compared devices with chemokine gradient, devices without chemokine gradient, and devices without chemokine (FIG. 1G). Here, the study tested 3 PBMC donors and two devices per donor, per conditions. Quantitative analysis of CXCR4+ cell polarization in devices with chemokine gradient indicated consistent polarization of CXCR4+ cells towards high CXCL12 side of devices, across 3 donors and in between device replicates. In contrast, the polarization was stochastic in devices without chemokine gradient, and devices without chemokine, across donors and within devices with same donors.

The presence of CXCL12 chemokine gradient increased the production of IgG1 antibodies in the supernatant (FIG. 1H), devices without chemokine gradient, and devices without chemokine. In contrast, the cytokine production was influenced by the presence of CXCL12 regardless of gradient. The chemokine gradient significantly increased the production of cytokines IL6, IL12p70, TNFα, IFNγ, IL2, IL10, IL-17, APRIL, and TNFb as compared to devices without chemokine (FIG. 1I). The devices with chemokine, without gradient were mostly higher in production of cytokines than devices with chemokine gradient. While the CXCL12 presence as a chemokine is a natural in vivo process, it is not easily possible to test in vivo the impact of gradient. The devices possibly allude to new insights into the regulatory role a gradient may play in cytokine secretion from activated B cells.

It has recently been described that CD4 T cells also use CXCR4 to localize to the dark zone [64]. Since the study used whole PBMCs, additional imaging experiments were performed and it was indeed observed that CD4+ T cells localized in the CXCR4 polarized spaces towards CXCL12 high portions of devices (FIG. 1J), suggesting that the lymphoid-on-chip recapitulates another aspect of in vivo GC biology.

### Materials and Methods

Polymers and peptides. PEG-4MAL with 10 kDa and 20 kDa molecular weight and >95% purity determined by NMR were purchased from Laysan Bio (catalog numbers 4arm-PEG-MAL-10k-1g, 4arm-PEG-MAL-20K-5g). PEG-4VS with 20 kDa molecular weight and >90% purity was purchased from Sigma Aldrich (catalog number JKA7025-1G). Peptides (>95% purity) were custom purchased from AAPPTec and included collagen I mimic ‘GFOGER’ (GYGGGP(GPP)5GFOGER(GPP)5GPC, where O=hydroxyproline), fibronectin/vitronectin mimic ‘RGD’ (GRGDSPC), VCAM1 mimic ‘REDV’ (GREDVGC), control scrambled peptide (GRDGSPC) and protease-sensitive crosslinker (GCRDVPMS↓MRGGDRCG). The non-degradable crosslinker DTT was purchased from Sigma Aldrich (catalog number DTT-RO). All components were reconstituted in 0.01 M HEPES (ThermoFisher, catalog number 15630080), pH 7.4.

Synthetic hydrogel-based organoid fabrication. Organoids were formed in synthetic hydrogels made of PEG-4MAL (or PEG-4VS) with typically a 7.5% (w/v) macromer concentration. In some experiments, organoid biophysical parameters were modified by varying PEG-4MAL w/v % or the ratio of adhesive peptide to crosslinker. PEG-4MAL was first functionalized at pH 7.4 with thiolated adhesive peptides RGD, REDV, GFOGER, or control peptide REVD or RDG for 30 min at 37° C. While RGD and REDV were used at 3.7 mM concentration, due to size constraints, GFOGER was functionalized at lower molarity (1 mM) and remaining 2.7 mM were either RDG (in GFOGER groups) or RGD (in GFOGER+RGD groups). Protease-sensitive crosslinker (VPM) and non-degradable crosslinker (DTT) were combined at a 1:1 molar ratio and adjusted to pH 6. Briefly, before combining the polymer and crosslinker, cell mixtures were suspended in the crosslinker solution. For tonsil organoids fabrication, cell mixtures included 200,000 tonsil mononuclear cells with 40,000 CD40L-TCM stromal cells. For PBMC organoids fabrication, mixtures included 400,000 cells with 40,000 CD40L-TCM stromal cells. After mixing, cell-crosslinker solutions (10 μL) were immediately injected into an equal volume of functionalized PEG-4MAL located in a well of a non-treated 96-well plate, making up a total of 20 μL. The ensuing droplet was mixed via pipetting and cured for 15 min at 37° C. Appropriate media was added post-incubation to begin organoid cultures. Hydrogel-based organoids were cultured in Roswell Park Memorial Institute (RPMI) 1640 medium (ThermoFisher, catalog number 11875119) supplemented with 10% FBS, 1×NEAA (ThermoFisher, 11140-050), 1× Sodium Pyruvate (ThermoFisher, 11360-070), 1× Penicillin-Streptomycin (ThermoFisher, 15140163), 1× 2-mercaptoethanol (ThermoFisher, 21985-023), 100 ng/mL human BAFF (R&D Systems, 2149-BF-010), 20 ng/mL human IL4 (Peprotech, 200-04-100UG) for first 2 days, and 10 ng/mL human IL21 (Peprotech, 200-21-100UG) for the rest of the culture. The media was replenished every 2 days. The organoids were challenged with either 10 μg/mL inactivated New Caledonia 20/99 (H1N1) influenza A virus (Biorad, PIP021), or 10 L/mL 2022-2023 Fluzone vaccine, with or without 0.5 μM R848 diluted in culture medium.

Viral Infection and FDC Imaging. For viral infection, PEG-4MAL Organoids were fabricated using PBMCs, HK-FDCs, and CD40L-TCM cells. After hydrogel formation at 37° C., media containing the live H1N1 (A/PR/8/34) virus was added to induce viral infection in cultures. 10⁶ CEID₅₀ virus units were added to each organoid. Media was replenished every two days. Inactivation of virus was performed for equivalent viral particles at 56° C. for 30 min, as reported earlier [73], and the added to the organoid cultures. Organoids were washed with PBS and fixed with 4% paraformaldehyde. After fixation, organoids were washed with PBS and permeabilized with 0.1% Tx-100 for 30 min and washed twice with PBS. Blocking was performed with 5% BSA for 90 min. Primary antibody was added to target at viral nucleoprotein (1:1000, Genetex, GTX14213) at 4° C. overnight and anti-mouse AF647 IgG2a secondary antibody (1:100, Biolegend, 407116) for 3 hours at RT was completed and unbound antibodies were washed out twice with phosphate-buffered saline solution with 0.02% w/v Tween™ 20. Further staining was performed with AF488 conjugated anti-human CD20 (1:100, Biolegend, 302316) and DAPI. Samples were washed with PBST twice and stored in PBS at 4° C. until imaging.

For FDC imaging, organoids were fabricated using CD40L-TCM, pre-stained HK-FDCs, and pre-stained PBMCs. The PBMC were pre-stained with green cell tracker CMFDA dye (1:1000, Invitrogen, C7025) for 30 min at 37° C. HK-FDCs were stained with Cell tracer violet (1:500, Invitrogen, C34557a) for 30 min at 37° C. Cells were washed twice with PBS to remove the unbound stain. After hydrogel formation, appropriate media was added to begin the culture. Organoids were treated with AF555 conjugated inactivated H1N1 antigen in presence of adjuvant R848 for 2.5 hours at 37° C., 5% CO2. After incubation, organoids were washed with PBS thrice and fixed with 4% paraformaldehyde for 30 min. After fixation, organoids were further washed twice with PBS and stored in the fridge until imaging.

Antigen conjugation procedure. H1N1 virus (Biorad, PIP021) was conjugated with anti-H1N1 A antibody (1:1000, Genetex, Cat #GTX40513), and anti-goat IgG (H+L) AF555 (1:100, ThermoFisher, A-21432) for 30 min at room temperature.

Flow cytometry. At the end of the desired culture time, organoids were washed with PBS and enzymatically digested in 150 U/mL type I collagenase (Worthington Biochemical, catalog number LS004194) for 1 h at 37° C. Buffer-containing serum was added at the end of this incubation to terminate the enzymatic activity. Organoid debris was removed using 96-well MultiScreen Mesh Filter Plates (EMD Millipore, catalog number MANMN6010), and the ensuing cells were resuspended in FACS buffer (PBS with calcium and magnesium, 2% FBS, 1% penicillin G/streptomycin and 5 mM EDTA). Cells were first stained with antibodies against cell surface antigens and incubated in the dark on ice for 1 h. For intracellular antigens, cells were then fixed and permeabilized using the eBioscience Foxp3/Transcription Factor staining buffer (ThermoFisher, catalog number 00-5523-00) set. After fixation, cells were incubated in a permeabilization buffer with antibodies against intracellular antigens for 1 h on ice. Cells were analyzed using a BD Fortessa flow cytometer. Data were analyzed using FlowJo software (10.9.1) using positive and negative controls; gating strategies are shown in the Figures. The following antibodies were used: CD19 (1:200, BD, 740287), CD38 (1:200, Biolegend, 303510), CD27 (1:200, Biolegend, 124216), CD86 (1:200, BD, 562432), CD184 (BD, 740799), IgM (1:200, Biolegend, 314524), IgG (1:200, Biolegend, 410720), CD138 (1:200, BD, 552026), Live/Dead (1:200, Thermofisher, L34962A). TSNE-2D TSNE embedding was generated using open-t-SNE (v0.7.1) from single-cell flow cytometry data with the following marker set: CD38, CD19, CD86, CXCR4, CD138, CD27. Single-cell expression levels are z-score and batch-normalized to take into account sample variability using Scanorama (v1.7.3). t-SNE generated with perplexity=50 and affinity=50. Single-cell phenotype extracted from FlowJo gating.

Organoid imaging. For confocal imaging, organoids were fabricated and cultured in glass-bottom 96-well plates (Cellvis). After up to 24 days in culture, organoids were fixed in 4% paraformaldehyde in PBS (Thermo Scientific Chemicals) for 30 min. Organoids were permeabilized with 0.5% Triton-X-100 in PBS for 30 min and blocked with 10% normal goat serum (Abcam) in PBS for 1 hour. Organoids were incubated with primary antibodies overnight. After washing with PBS-Tween, organoids were incubated with fluorescence-conjugated secondary antibodies and nuclear stain (DAPI) for 1 hour. Organoids were then washed and resuspended in PBS and imaged on a Zeiss LSM900 confocal microscope. Primary antibodies used for staining include anti-CD83 (1:100, Sigma, HPA041454-100UL), anti-CXCR4 (1:100, Abcam, ab181020), anti-CD20 (1:50, ThermoFisher, 50-0202-82), anti-BCL6 (1:100, Abcam, ab220092), anti-AID (1:100, ThermoFisher, 39-2500). Secondary antibodies include anti-mouse IgG (H+L) AF488 (1:100, ThermoFisher, A-11001), anti-rabbit IgG (H+L) AF555 (1:100, ThermoFisher, A-21428), anti-goat IgG (H+L) AF555 (1:100, ThermoFisher, A-21432).

Tonsil & PBMC Patients Information:

| | |
|---|---|
| Age | Sex |

Tonsil Donors

| | | |
|---|---|---|
| Patient 4 | 13 | Female |
| Patient 7 | 9 | Female |
| Patient 8 | 2 | Male |
| Patient 9 | Confirming | Confirming |
| Patient 10 | 11 | Male |

PBMC Donors

| | | |
|---|---|---|
| 802C | 26 | Female |
| 811C | 20 | Male |
| 902C | 22 | Male |
| 003 | 21 | Male |
| 301C | 27 | Female |

ELISPOT. Organoids cultures that were either stimulated for 2 days with inactivated virus (or Fluzone) or left unstimulated were degraded and plated on inactivated virus-coated (Bio-Rad, PIP021) and blocked 96-well PVDF membrane plates (Millipore, MSIPS4510). Cells were incubated on these membranes, undisturbed overnight at 37° C. Plates were washed and treated with horseradish peroxidase-conjugated anti-IgG/IgA/IgM (1:5000, ThermoFisher, A24494) secondary antibody. After incubation overnight at 4° C., plates were washed and developed with AEC substrate (BD), washed 5 times with water, dried, and spots were enumerated with a stereomicroscope and Matlab (2021b).

Device fabrication and microfluidic device setup. Devices made of poly-dimethyl siloxane (PDMS) were fabricated using Sylgard 184 (24236-10, Electron Microscopy Sciences) with a weight ratio of 10:1 (base agent: curing agent). After mixing of base agent and curing agent, the PDMS mixture was vacuumed to remove bubbles. PDMS mixture was then poured over a silicon wafer with the microfluidic device design and left in a 65° C. oven overnight. Devices were detached from the mold and 4 mm diameter holes were punched using a biopsy punch. Devices were then oxygen plasma bonded to a clean glass slide using a plasma chamber (PDC-001-HP, Harrick Plasma) for 35 secs at the highest power setting. Once bonded, devices were left in the 80° C. oven for up to 1 week prior to use for the experiment. Bonded devices were treated using 1% poly-L-Lysine solution to facilitate adherence of PEG-4MAL hydrogels to the surface of the device prior to placing PEG-4MAL hydrogels in the device wells. Devices with hydrogel were then connected to two inlet lines containing media that flowed at a rate of 1.0 L/min using a syringe pump (PHD Ultra, Harvard Instruments). In devices treated with CXCL12, one syringe was replaced with media containing 100 ng/mL of CXCL12 after 2 days in culture. Devices were inverted over a Petri dish for media collection for 8-12 days.

Rheology testing. PEG hydrogels were formed in 10 uL volume on parafilm and upon gelation at 37° C., were transferred into a 12-well plate and swelled in 1×PBS overnight. Fully swollen hydrogels were then tested using a rheometer (MCR-302, Anton Paar; CP10-2) at 37° C. A frequency sweep (10 to 0.1 rad s−1) was performed at a constant strain of 2%. Storage (G′) and loss (G″) moduli were determined by averaging all data points acquired from 10 to 0.1 rad/s interval.

Imaging mass cytometry. The antibodies used for imaging mass cytometry (IMC) was either bought pre-conjugated to unique metal tags from Standard BioTools or bought in a carrier-free format and conjugated in-house. The conjugation reaction involved loading a Maxpar X8 polymer with a unique lanthanide metal. Simultaneously, the purified antibody is partially reduced using Tris(2-carboxyethyl)phosphine (TCEP). After competing for the two reactions, the antibody is mixed with the metal-loaded polymer for the conjugation reaction [74]. The final yield for the conjugated antibodies ranged between 50-70% of the initial starting amount of the antibody.

The tonsil tissues used in this study are formalin-fixed, paraffin-embedded (FFPE) with a thickness of 5-μm which is within the tissue thickness recommended for IMC (≤7-μm). It was obtained from a third-party vendor, TissueArray.com under the tissue ID HuFPT161. Standard BioTool's IMC staining protocol was used in this study and it was also used in previously published papers [75] [76]. The protocol starts with baking the tissues at 60° C. followed by deparaffinization using xylene immersion and rehydration using descending concentrations of ethanol. The heat-induced epitope retrieval method was used using Dako's target retrieval solution (Catalog number: S2367, Agilent Dako) with pH=9. The protein blocking was then performed using Dako's ready-to-use blocking buffer solution (Catalog number: X090930-2, Agilent Dako). The antibody cocktail mix was prepared in the protein-blocking buffer and incubated with the samples overnight at 4° C. The following day, the samples are then stained with Intercalator-191Ir/193Ir for nuclear counterstaining. The samples were then left to dry overnight before imaging.

Serial sections stained with H&E (hematoxylin and eosin) were used to annotate the areas with follicles that were later used for IMC imaging. The Hyperion Imaging System was first tuned using 3-Element Full Coverage Tuning Slide (PN 201088) to optimize helium flow. Laser power was then tested on smaller test regions to choose the optimal laser power with an acceptable S/N ratio. The acquired data gets automatically saved in .mcd format which is later viewed using Standard BioTools' MCD Viewer software (v 1.0.560.2). Single-channel images were then exported from all tissues in the .ome-tiff format.

RNA-FISH. The HCR staining follows the protocol provided by Molecular Instruments RNA-FISH on mammalian cells on a slide. The immune organoids were first fixed using 4% PFA fixation buffer (mixed 10 mL 16% Paraformaldehyde (PFA, Cat #28908), 4 mL 10×PBS (REF 70011-044), 26 mL RNase free water (Cat #10977-015) for 40 mL of buffer) for 15 min at room temperature. Fixed immune organoids were permeabilized in 70% ethanol at −20° C. overnight. After permeabilization, the organoids were air-dried for 15 min and washed with 2×SSC buffer 3 times. The samples were then pre-hybridized in 50 μL pre-warmed HCR probe hybridization buffer per organoid at 37° C. for 30 min. 0.5 μL of HCR probes for desired targets were then added to 50 μL of pre-warmed probe hybridization buffer. The prehybridization buffer was aspirated, and the probe dilute was added to the samples. The samples were incubated at 37° C. overnight. The samples were then washed with 75 μL of pre-warmed probe wash buffer per organoid for 5 min at 37° C. 4 times. Then, the samples were washed with 100 μL of 5×SSCT (5×SSC, 0.1% Tween 20) per organoid for 5 min at room temperature twice. The samples were then pre-amplified with 50 μL of amplification buffer per organoid at room temperature for 30 min. 1 μL of each HCR amplifier hairpin was then snap-cooled to 95° C. for 90 sec and cooled down to room temperature in a dark drawer for 30 min. The snap-cooled hairpins were then added to 50 μL of amplification buffer. The pre-amplification buffer was then aspirated, and diluted hairpins were added to the sample. The samples were then incubated overnight. The amplification solution was then aspirated, and the samples were washed with 5×SSCT for 5 min at room temperature 5 times (see HCR protocol for cells on slide). 100 μL of antifade mounting buffer per organoid was added for imaging. The antifade mounting buffer containing Tris-HCL (20 mM), NaCl (50 mM), glucose (0.8%), saturated Trolox (Sigma, 53188-07-1), pyranose oxidase (Sigma: P4234), and catalase (Sigma, 9001 May 2, 1:1000 dilution). DNase I (Sigma, 04716728001) was used to remove the fluorescent signal. After imaging, the samples were first washed with 2×SSC twice. We then diluted 50 μL of 10× concentration incubation buffer in 450 μL of RNase-free water to 1× concentration. The samples were then incubated with the 1× incubation buffer at room temperature for 5 min. Then 10 μL of DNase I, 50 μL of 10× incubation buffer, and 440 μL of RNase-free water were mixed to make the DNase I mixture. The samples were then incubated in the DNase I mixture for 4 hours at room temperature. After incubation, the samples were then washed 3 times with 30% formamide buffer at room temperature for 5 min. Samples were then imaged again to confirm the removal of the RNA-FISH signal. The samples were then ready to start the next cycle of RNA labeling. The IF staining is conducted after all RNA labeling is completed.

Multiplex Immunoassay. Organoid culture supernatant samples were analyzed for influenza-specific IgG binding using the V-PLEX Respiratory Panel 1 (Catalog No. K15365U). This assay is based on an electro-chemiluminescent-based multiplex immunoassay provided by Mesoscale Discovery (MSD-ELICA). Experiments were done largely according to the manufacturer's instructions. Plates were coated with the influenza HA antigens such as A/Hong Kong/4801/2014 (H3N2), A/Michigan/45/2015 (H1N1) pdm09, A/Shanghai/1/13 (H7N9), B/Brisbane/60/2008 (Influenza B), B/Phuket/3073/2013 (Influenza B). Blocking was done with 150 □L of MSD Blocker A. To assess binding, samples were diluted and 50 □L of each sample was added to the plates in duplicate. Following this, 50 □L of 1×MSD SULFO-TAG Anti-Human IgG detection antibodies were added to each well. Following the detection reagent step, 150 □L per well of MSD Gold Read Buffer B was added to each plate immediately prior to reading on an MSD plate reader (MESO QuickPlex SQ 120). Plates were washed three times with 300 □L PBS/0.05% Tween between each step. For each incubation, plates were kept for shaking at a speed of 700 rpm and at room temperature. IgG signal data was analyzed using Discovery Workbench and Prizm software.

Stiffness measurement of tonsil tissue. Samples were analyzed using atomic for microscopy (AFM, MFP-3D, Asylum Research). Beaded silicon nitride cantilevers (4 m polystyrene bead, spring constant of 30.5 pN per nm) were prepared and used to indent the surface of the tissue. Tissue specimens were adhered to a glass substrate at ambient temperature and immersed in PBS to maintain a hydrated state. Immediately prior to the force mapping, the AFM cantilever was calibrated using the glass bottom of a FluoroDish to determine the deflection inverse optical lever sensitivity. The cantilever was then raised away from the glass surface to monitor the thermal vibrations and assess the cantilever's spring constant using the thermal vibration method by fitting the thermal spectrum to a Lorentzian function. All samples were measured with a force map with a cantilever deflection of 5 m. With an approach rate of 5 m per second. A Hertzian model was used to compute Young's modulus of the tissue fitting to the force indention curves [77]. Each tonsil had its capsule and internal tissue measurements collected on the same day.

Statistics & Reproducibility. All studies were replicated 3 times and results were consistent across independent experimental runs. Investigators were blinded during imaging analyses, flow cytometry experiments, and formulation tests. Blinding was performed without providing information on the type of organoids or treatment groups. Sample sizes of organoids were selected based on statistical power calculations and previous experience with these metrics [30] [45] [78] [79 [80]. No data from intact hydrogels were excluded. All statistical analysis was performed using GraphPad Prism 9 software. Data is generally presented as mean±SD unless otherwise noted, with figure legends indicating specific statistical tests. all Data was tested formally for normality or statistical assumptions, and where necessary statistical corrections were applied to address unequal standard deviations or heteroscedasticity.

Imaging Data analysis. For quantification of the CD83 marker clusters, first the images were downsampled by averaging the intensity in patches of 5 by 5 pixels. A threshold of 25 was defined to extract positive regions for CD83 marker. Each separate mask region was considered to be a unique cluster. We measure the average area in terms of pixels for positive regions, the total number of unique clusters, and the average number of pixels per unique cluster. For orientation analysis, first the images were downsampled by averaging the intensity in patches of 5 by 5 pixels and then the region mask of each organelle was extracted by thresholding the maximum projection of all markers. Then, each organelle image was divided into left and right regions by considering positive and negative regions on the x-axis with the zero defined as the organoid mask centroid. The image orientation is defined as the gradient of chemokines from the left side to the right side of the images. Using an intensity threshold of 25, the study could extract the positive region for CXCR4. The proportion of positive regions was calculated in the left and right regions respectively.

### Example 2: Rational Design of Synthetic Tonsil Organoids

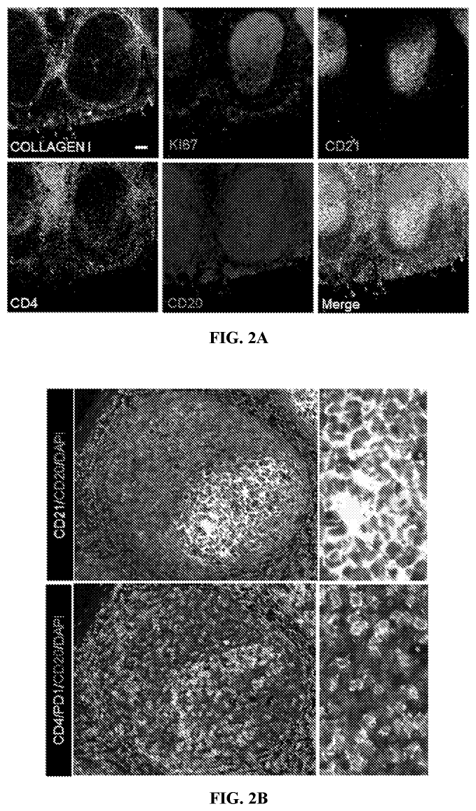

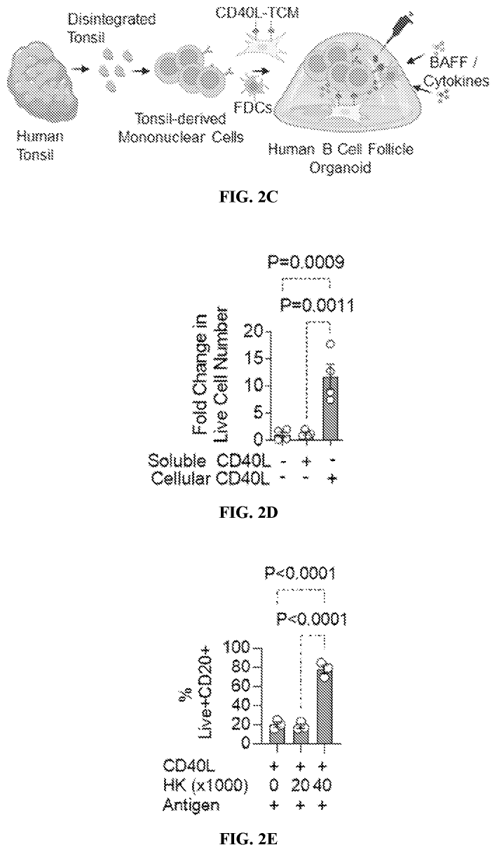

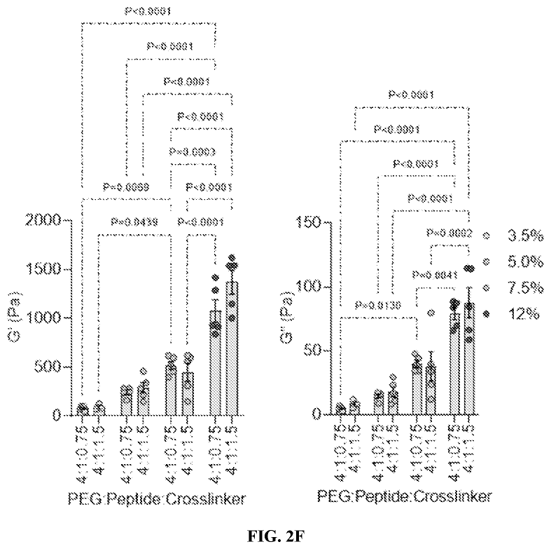

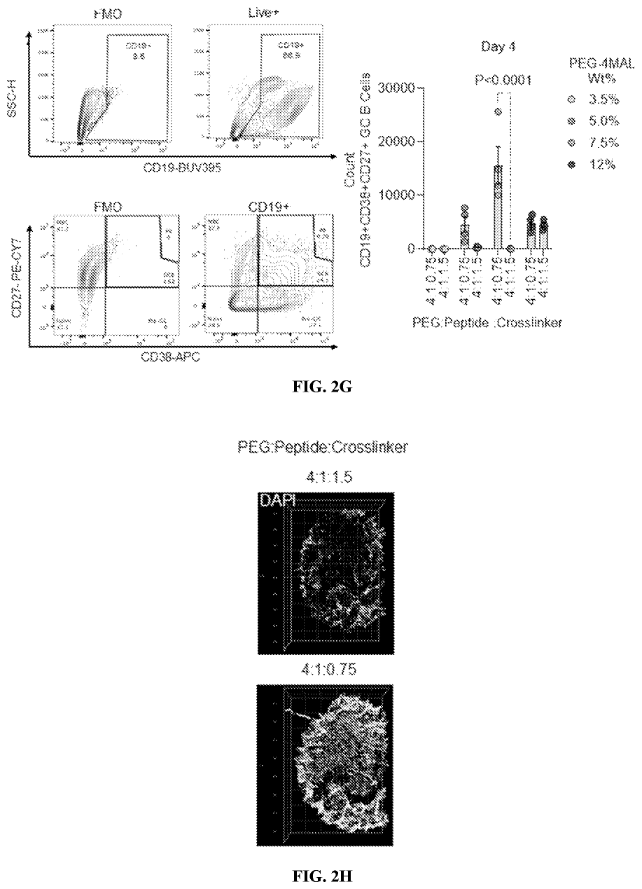

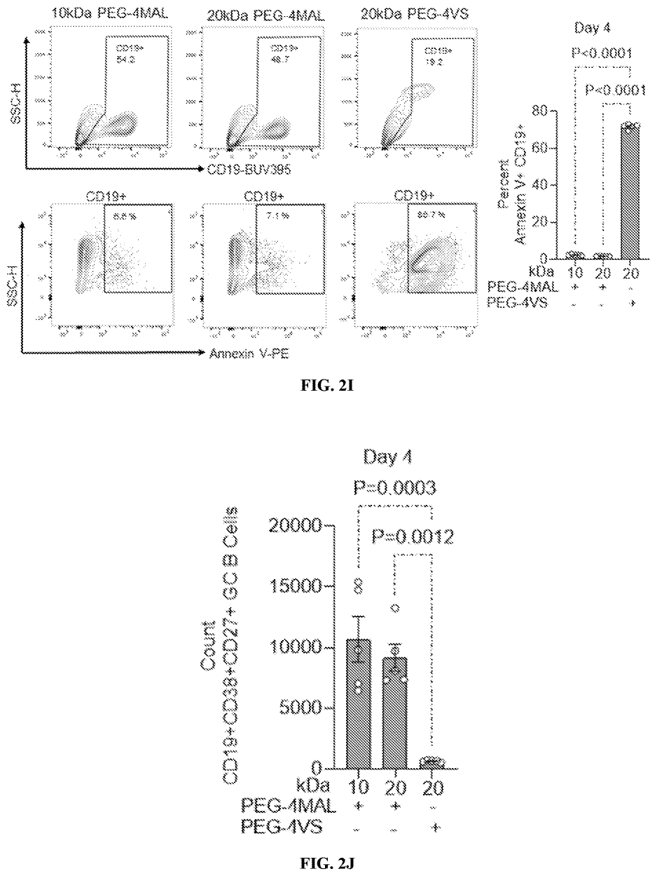

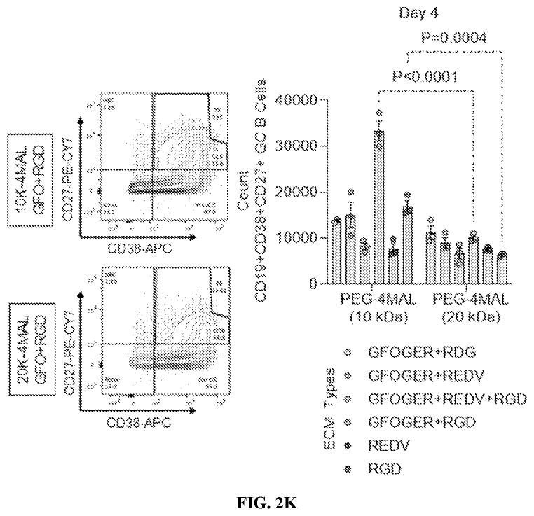

The microenvironment of lymphoid organs supports immune function through its structural, cellular and molecular composition. To establish the key components of tonsil lymphoid tissues, a study was conducted which performed imaging mass cytometry and observed the presence of extracellular matrix (Collagen 1+), T cells (CD4+), B cells (CD20+), proliferative B cells (CD20+Ki67+), and follicular dendritic cells (FDCs, CD21+) (FIG. 2A). The B cells were found in the vicinity of follicular T helper cells (PD1+CD4+) (FIG. 2B) and FDCs (FIG. 2B). The B cells interact with stromal cells, such as FDCs, and follicular T helper cells through multiple receptor-ligand interactions and through secreted cytokines and chemokines. In vivo, the presence of antigen activates CD4+ T cells to differentiate into follicular T helper cells that express CD40L, which engages with CD40 on B cells to initiate GC reactions. While the antigen can directly bind to B cells and can be presented through FDCs, integrin-mediated cell-cell adhesion also plays a central role, Integrins are differentially expressed by naïve and CC B cells. Integrin α4β1 (very late antigen-4) expression plays a key role in stabilizing the initial interaction of B cells with antigens associated on the surface of FDCs that express its ligand, Vascular cell adhesion protein 1 (VCAM-1), which in turn enables B cell activation. In contrast to activated CC B cells in human tonsils, the naïve B cells express higher levels of CD49d (α4) and CD29 (β1)22. Current state-of-the-art human tonsil transwell aggregates lack controlled integrin-ligand-mediated microenvironment interactions found in B cell follicles and do not present stromal or T-cell signals in a controlled manner. These systems also do not possess the multitier soft tissue mechanical properties of lymph nodes, as reported earlier. Therefore, the study engineered a synthetic hydrogel to present VCAM-1-mimicking peptides through its polymeric arms. A 4-arm polyethylene glycol, end-functionalized with Maleimide groups (PEG-4MAL) was biofunctionalized with thiolated, cysteine-flanked, VCA M1-mimicking REDV peptides at 3.0 mM to maximize integrin-mediated adhesion sites. These hydrogel macromers were crosslinked with a mixture of protease degradable crosslinker peptide (GCRDVPM SMRGGDRCG, referred to as VPM) and non-degradable crosslinker dithiothreitol (DTT), previously optimized to provide stability to B cell cultures. To test whether T cell signal CD40L could be presented as a soluble recombinant protein for B cell activation, the study compared soluble CD40L (100 ng/mL) to CD40L-expressed on an engineered fibroblast (a T cell mimic (CD40L-TCM)) that was recently reported. A mononuclear fraction of 200,000 freshly isolated tonsil cells were encapsulated with soluble CD40L or CD40L-TCMs in 7.5 wt % PEG-4MAL hydrogels (FIG. 2C), based on recent work with human B cell lymphomas. The hydrogels were developed in a 96-well plate and in the initial set of optimization studies, human IL-4 (20 ng/mL) with BAFF (100 ng/mL) was added for 4 days. Flow cytometry analysis indicated that CD40L presented in its membrane-bound format as CD40L-TCM increased the viability of total tonsil mononuclear cells as compared to soluble CD40L (FIG. 2D).

The study next investigated the role of human FDCs on B cell expansion. FDCs are a specialized type of antigen-presenting dendritic cells found in B cell follicles, and their primary function includes maintaining the follicular architecture, binding and retaining antigens through immune complexes, secreting chemokines, and presenting these antigens to B cells for GC reaction. FDCs also rescue antigen-bound B cells from apoptosis. Therefore, the study incorporated human tonsil-derived FDC, called HK cells, and showed that number of live tonsil mononuclear cells increased in an FDC density-dependent manner, with the maximum response obtained at 40,000 FDCs per hydrogel (FIG. 2E). Notably, both CD40L and FDC presence was needed to sustain the survival of CD20+ B cells.

GC B cells in follicles are diffused aggregates of specialized B cells that undergo rapid proliferation. To generate a polymer network of PEG-4MAL that allowed for cell proliferation and aggregation, the study tested two crosslinking densities by using (PEG-4MAL):(adhesive-peptide):(Crosslinker) ratios of 4:1:1.5 and 4:1:0.75, and varied hydrogel stiffness using different weight % of PEG-4MAL. The study results did not show a significant difference between the crosslinker ratios or polymer wt %, except for when the wt % was increased to 12%. The resulting storage modulus at 7.5% was 450-520 Pa, whereas that at 12% was 1000-1370 Pa (FIG. 2F). The gels at 3.5 and 5 wt % were much softer, with storage modulus in the 75 to 290 Pa range (FIG. 2F). The loss moduli of the gels followed a similar trend and suggested that the gels behaved as elastic solids (FIG. 2F). These rheological results are comparable to those obtained from hydrogels fabricated with fixed adhesive peptide densities; therefore ratiometric approach was chosen. The 4:1:0.75 hydrogel composition at 7.5 wt % led to a significantly higher number of GC B cells (live+CD19+CD27+CD38+) and plasmablasts (PB: live+CD19+CD38++CD27+) (FIG. 2G). The high cell numbers in lower crosslinking density gels were further supported by confocal imaging (FIG. 2H). Lastly, the study tested whether encapsulation of cells in hydrogels would impact the viscoelastic properties of the gels, and indeed the storage modulus at 7.5% dropped by 100 Pa over 4 days but remained the same thereafter till at least 12 days of culture.

To achieve high viability of B cells, the study compared 10 kDa PEG-4MAL, 20 kDa PEG-4MAL, and 20 kDa 4-arm PEG-vinyl sulfone (PEG-4VS) using the same concentration of REDV peptide and crosslinking density. PEG-4VS, with a slower Michael-type addition gelation kinetics than PEG-4MAL, has previously been reported for the development of bioadhesive, protease-degradable hydrogels. These hydrogel conditions had similar storage modulus and therefore represented a reasonable testbed for understanding the effect of PEG-based formulations. Among the three macromers, the study results showed that PEG-4VS showed a 20-fold higher percentage of apoptotic B cells than those of the other two PEG-4MAL macromers (FIG. 2I). Additionally, the study examined the GC response induced in each macromer by looking at the GC B cell phenotype (Live+CD19+CD38+CD27+). PEG-4VS displayed a 15-fold lower number of GC B cells than the other two macromers over 4 days of culture (FIG. 2J).

While VCAM-1 is an important cell-based signal and integrin α4β1 is expressed on B cells, integrin αvβ3 is expressed abundantly in tonsillar T cells, and imaging of human tonsil tissues indicates the presence of collagen type I around B cell follicles, with partially interspersed collagen within the follicles around the proliferative B cells (Ki67+) (FIG. 2A). To understand the effect of integrin-binding ligands on human GC B cell induction in the organoids, the study used functionalized PEG-4MAL hydrogels with different bioadhesive peptide combinations: ‘REDV’, fibronectin mimic ‘RGD’ (GRGDSPC), and collagen-1-mimicking peptide ‘GFOGER’ (GYGGGP(GPP)₅GFOGER(GPP)₅GPC, where O=hydroxyproline). GFOGER binds to multiple β₁ integrin diners, including integrin α₂β₁. Specific combinations included GFOGER+RGD, GFOGER+RDG (a scrambled peptide), GFOGER+REDV, GFOGER+REDV+RGD, REDV, and RGD, all used at a final 3 mM peptide concentrations. The study results showed that the combination of peptide ligand combinations and PEG-4MAL molecular weight had a distinct impact on GC phenotype development. The combination of RGD and GFOGER led to a 2-fold increase of GC B cells and plasmablasts compared with RGD alone or GFOGER alone in 10 kDa PEG-4MAL hydrogels (FIG. 2K). The combinatorial effect was significantly higher than that induced in hydrogels functionalized with RGD or REDV or GFOGER alone. These findings indicate that a combination of 10 kDa PEG-4MAL and GFOGER+RGD matrix induced significantly higher numbers of GC B cells compared to other groups.

Discussion. The microenvironment of lymphoid organs supports immune function through its structural, cellular and molecular composition. To establish the key components of tonsil lymphoid tissues, imaging mass cytometry was performed and showed the presence of extracellular matrix (Collagen 1+), T cells (CD4+), B cells (CD20+), proliferative B cells (CD20+Ki67+), and follicular dendritic cells (FDCs, CD21+) (FIG. 2A). The B cells were found in the vicinity of follicular T helper cells (PD1+CD4+) (FIG. 2B) and FDCs (FIG. 2B). The B cells interact with stromal cells, such as FDCs, and follicular T helper cells through multiple receptor-ligand interactions and through secreted cytokines and chemokines. In vivo, the presence of antigen activates CD4+ T cells to differentiate into follicular T helper cells that express CD40L, which engages with CD40 on B cells to initiate GC reactions. While the antigen can directly bind to B cells and can be presented through FDCs, integrin-mediated cell-cell adhesion also plays a central role. Integrins are differentially expressed by naïve and GC B cells. Integrin α4β1 (very late antigen-4) expression plays a key role in stabilizing the initial interaction of B cells with antigens associated on the surface of FDCs that express its ligand, Vascular cell adhesion protein 1 (VCAM-1), which in turn enables B cell activation. In contrast to activated GC B cells in human tonsils, the naïve B cells express higher levels of CD49d (α4) and CD29 (β1). Current state-of-the-art human tonsil transwell aggregates lack controlled integrin-ligand-mediated microenvironment interactions found in B cell follicles and do not present stromal or T-cell signals in a controlled manner. These systems also do not possess the multitier soft tissue mechanical properties of lymph nodes, as reported earlier.

### Example 3: Presentation of Tonsil ECM to Tonsil-Derived B Cells Enhances GC Reaction More than 2D Cultures

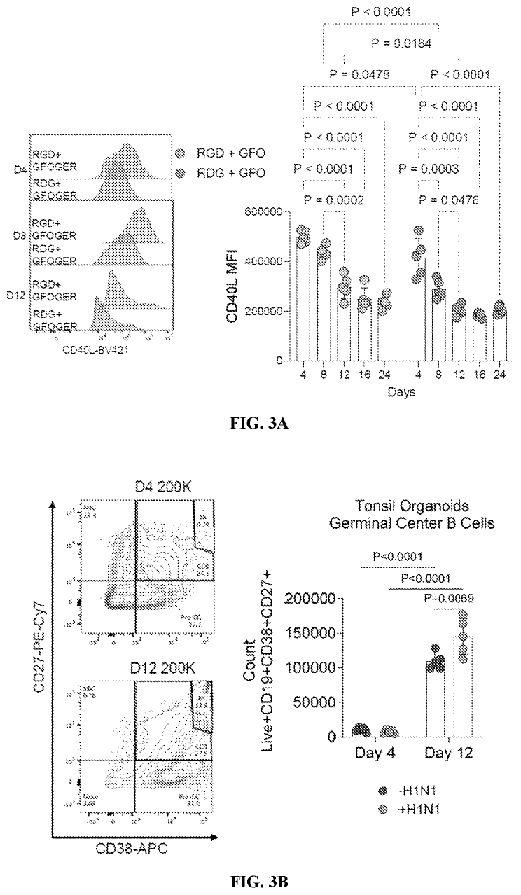

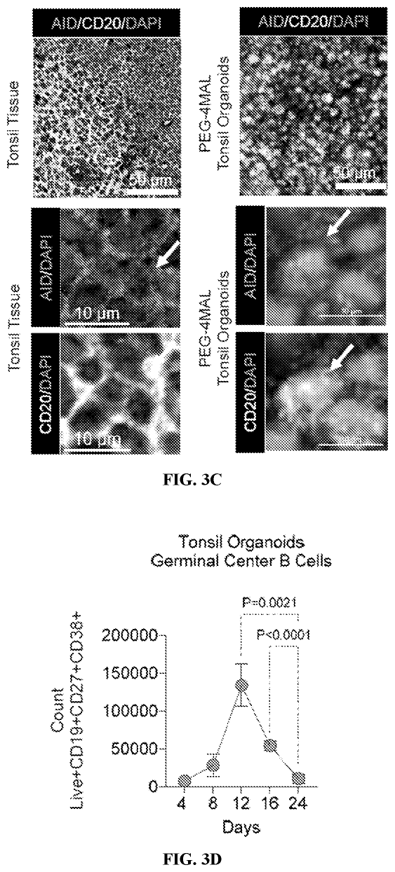

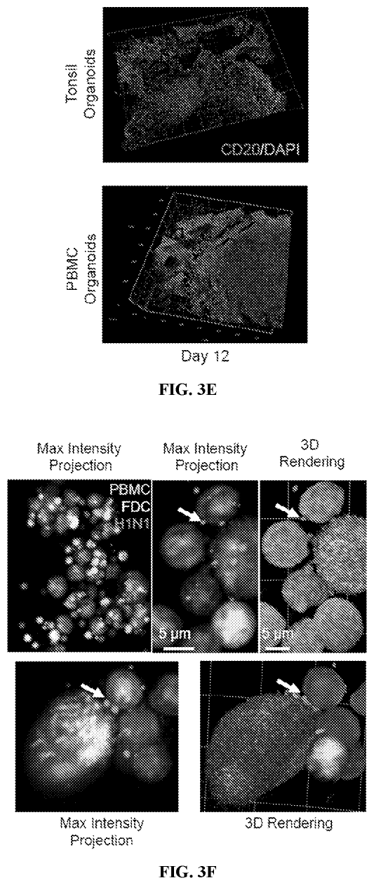

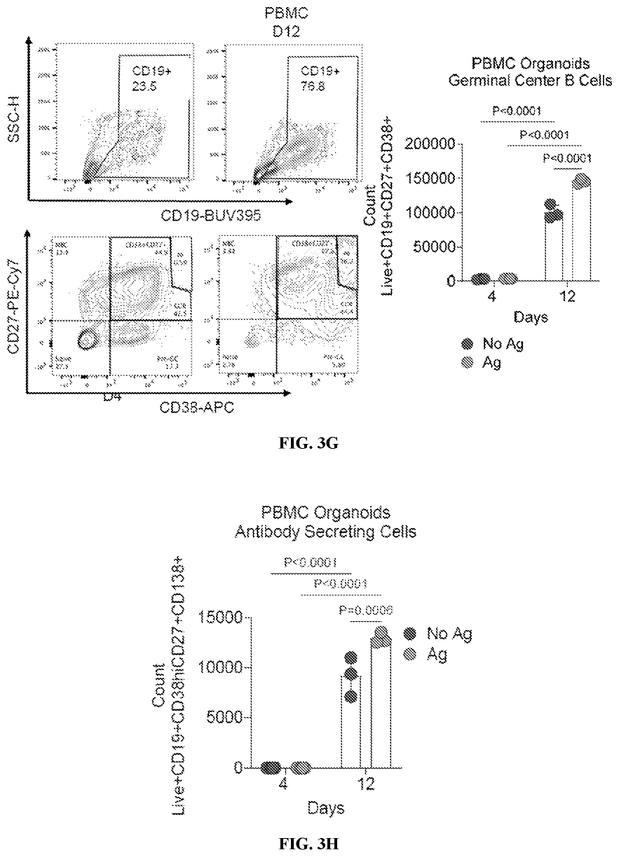

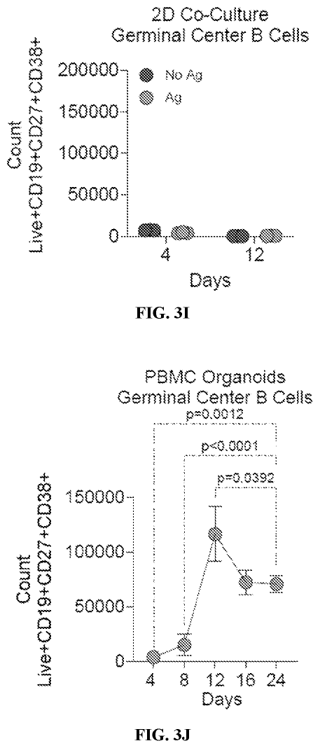

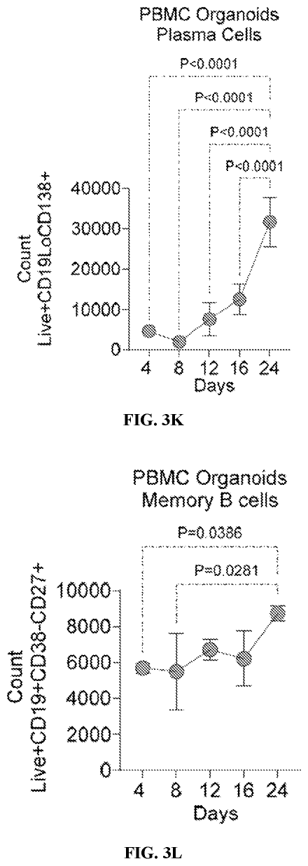

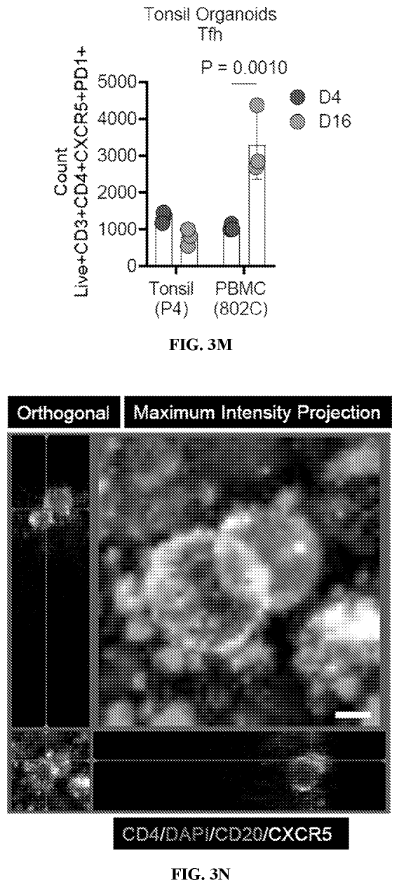

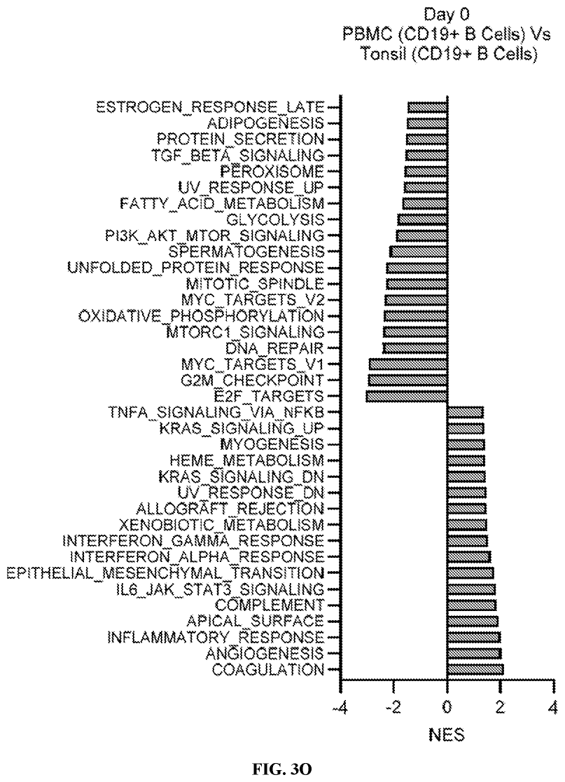

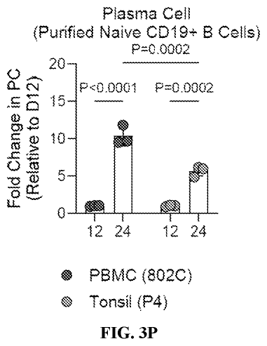

A study was conducted which determined whether the B cell response after antigen exposure within the tonsil organoids can be differentiated from those without any antigen exposure. In all these cultures, CD40L is presented as CD40L-TCM, however, the expression of CD40L significantly reduces by day 12, likely due to mitomycin C treatment (FIG. 3A). This is advantageous because in vivo the CD40L signal is transient, and it was expected that CD40L continued expression would reduce the progression of the GC reaction. With 200,000 tonsil cells seeded per 10 μL hydrogel, the study observed a 19-fold increase in the Live+CD19+CD38+CD27+GC B cells in H1N1 antigen-exposed organoids by day 12 as compared to 10-fold in no antigen groups (FIG. 3B). The antigen used was 10 μg/mL of formalin-inactivated New Caledonia 20/99 (H1N1) influenza A virus, added directly to the media in which hydrogels were incubated. The formation of GC B cells was further confirmed by the expression of hallmark GC marker, Activation-induced cytidine deaminase (AID), in B cells on day 12, with marked similarity to native tonsil tissues (FIG. 3C). The development of GC phenotype in the no-antigen control group is attributable to the presence of CD40L-TCM. In contrast, the Live+CD19+CD38hiCD27+CD138+ ASC and Live+CD19loCD138+ plasma cells were not significantly different between organoids exposed to antigen versus no antigen controls. We posit that this result reflects the intrinsic limitations of tonsil cells, as they are often inflamed or have prior exposure to microbes. The overall cell subtype-specific output on day 12 was markedly higher in PEG-4MAL-based tonsil organoids than in 2D coculture of CD40L-TCM and HK-FDCs with tonsil cells, in the presence of BAFF and cytokines, indicating a role for a 3D microenvironment that presents a multitude of biophysical properties in enhancing B cell differentiation. Upon extension of culture time for 3D immune organoids, the peak of the GC response in PEG-4MAL hydrogels was reached at 12 days followed by a rapid decrease in survival of GC B cells, ASCs, plasma cells, and memory B cells (FIG. 3D).

Arguably, the microenvironment profiling and PEG-4MAL results suggest Collagen I is a critical component of organoid culture. We compared PEG-4MAL-based tonsil-derived organoids with collagen-based tonsil cultures. Using commercially available collagen 1, the maximum storage modulus possible was 100 Pa, which was close to the PEG-4MAL 20 kDa stiffness observed. When tonsil mononuclear cells were encapsulated in collagen gels along with the same cellular composition as in PEG-4MAL gels, collagen gels did not support B cell survival and proliferation. The collagen gels were fragile and degraded before day 12 and yielded a considerably lower output of GC B cells and plasma cells.

### Example 4: Presentation of Tonsil Microenvironment to PBMCs Enhances Longevity of GC and Plasma Cells

A study was conducted which hypothesized that the presentation of the lymphoid microenvironment to PBMCs would lead to a more controlled B cell maturation response as they are less exposed to the inflamed environment of tonsils or microbes than cells derived from tonsil tissue. To test this, the study encapsulated human PBMCs in tonsil microenvironment-mimicking hydrogels with GFOGER+RGD ECM-mimicking peptides, FDCs, and CD40L-TCM. In the presence of IL4 and IL21, as outlined in tonsil-based organoids, PBMC-derived B cells formed similar 3D aggregates in PEG-4MAL-based organoids as observed in PEG-4MAL-based tonsil organoids (FIG. 3E). Furthermore, inactivated H1N1 virus antigen colocalized between human FDCs and B cells (FIG. 3F). This mimics the in vivo process whereby naive B cells in lymph nodes can encounter antigens in B-cell follicles either through direct binding of their immature B-cell receptors (BCRs) or on the surfaces of FDCs [22] [23]. These findings further show that viral antigens can diffuse through the hydrogel network and localize to encapsulated cells.

The Live+CD19+CD38+CD27+ GC B cell response in PEG-4MAL-based PBMC organoids increased over 12 days, along with a higher response for antigen-exposed conditions (GC B cell ~38-fold on day 12 compared to day 4) compared to the no-antigen control group (GC B cell 32-fold) (FIG. 3G). The GC response in the no-antigen group is attributable to the presence of CD40L and it has been previously shown that it leads to non-specific BCRs [45]. The Live+CD19+CD38hiCD27+CD138+ ASCs and Live+CD19loCD138+ plasma cell response also significantly increased in PEG-4MAL-based PBMC-derived organoids when exposed to inactivated H1N1 virus antigen (FIG. 3H). In stark contrast, in 2D culture, the number of GC B cells, ASCs, and plasma cells dramatically decreased over 12-day culture without proliferation (FIG. 3I). Importantly, the GC response was sustained with PEG-4MAL-based PBMC organoid culture (FIG. 3J) and the ex vivo number of plasma cells increased over 24 days (FIG. 3K). GC B cell numbers remained 19-fold higher than day 4 in PEG-4MAL-based PBMC organoids, in contrast to 1.3-fold high in PEG-4MAL-based tonsil organoids after 24 days. Similarly, plasma cell numbers increased by 6-fold in PEG-4MAL-based PBMC organoids by day 24 relative to day 4 as compared to a 50% drop in plasma cells in PEG-4MAL-based tonsil organoids. The Live+CD19+CD38hiCD27+CD138+ ASC counts increased by 550-fold in PEG-4MAL-based PBMC organoids by day 24 but increased only 57-fold in PEG-4MAL-based tonsil organoids by day 24.

Notably, in both tonsils and PBMC-derived organoids, the peak of GC and ASC responses occurred at 12 days, whereas the peak of plasma cells in PEG-4MAL-based PBMC organoids was on day 24 in contrast to day 12 in PEG-4MAL-based tonsil organoids (FIG. 3K). Because plasma cells differentiate after several rounds of GC B cell decision-making, it can be argued that a more accurate representation of the B-cell differentiation process is seen in PBMC-derived organoids where plasma cell response peaks after GCs and not in PEG-4MAL-based tonsil organoids where both GC and plasma peak at the same time. We also evaluated the differentiation of memory B cells (Live+CD19+CD38-CD27+) and observed that PEG-4MAL-based PBMC organoids sustained the memory phenotype over 24 days (FIG. 3L), whereas PEG-4MAL-based tonsil organoids peaked by day 12, followed by a sudden drop in numbers. t-SNE clustering analysis of flow cytometry markers indicated conservation of single cell population distributions across time in the PBMC organoids but not in the tonsil organoids.

Overall, the bioengineered PBMC-based human lymphoid organoids provide a strategy that provides a controlled presentation of the lymphoid microenvironment, leading to prolonged survival and does not rely on a tonsillectomy of inflamed tonsils from donors. The latter point is highly relevant as the PBMC organoids provide feasible, easy access from specific donors across various demographics, such as lymphoma patients, cancer patients, and patients with autoimmune disorders.

### Example 5: Immunostimulation Using TLR7/8 Agonist Increases the Immune Response Against H1N1 Vaccines

A limitation of inactivated virus-based vaccines is that the inactivation process in vaccine formulations either limits immune detection to only TLR7 sensing of viral RNA in plasmacytoid dendritic cells or the inactivation process can damage viral RNA due to cross-linking of the nucleic acids [46] [47], making the activation of pathogen recognition receptors (PRRs), including Toll-like receptor (TLR) 7 on B cells, difficult. Indeed, TLR7-deficient mice are not protected by inactivated influenza virus particles, and exhibit defects in antibody class-switching and plasma cell responses [48]. In contrast, live, infectious, influenza viruses can activate TLR7 on B cells. To compare the effectiveness of inactivated viruses in the preparation as vaccines to live viruses, the study exposed PEG-4MAL-based PBMC organoids with live H1N1 (A/PR/8/34) virus at 10⁶ CEID₅₀ (50% Chicken Embryo Infectious Dose) particle units. We observed that H1N1 virus particles colocalized with B cells and induced a significant increase in total B cells and GC B cell phenotypes in virus-infected cultures (at 10⁶ CEID₅₀) than in cultures treated with heat-inactivated H1N1 (A/PR/8/34) virus as an antigen at a dose equivalent to 10⁶ CEID₅₀. This observation prompted the investigation of the effect of TLR7-stimulating adjuvants with inactivated H1N1 viruses in organoids.

We supplemented inactivated H1N1 (A/PR/8/34) virus antigen with a chemical form of a TLR7/8 agonist, Resiquimod (R848), which is used as an adjuvant to induce strong Myd88-dependent activation of immune cells [49], including B cells [50] [51]. Organoids cultured with inactivated A/PR/8/34 H1N1 virus antigen (dose equivalent to live A/PR/8/34) and 5 μM TLR7/8 agonist exhibited a significantly higher number of Live+CD19+ B cells and Live+CD19+CD27+CD38+GC B cells per organoid after 12 days of culture as compared to R848 alone or inactivated A/PR/8/34 H1N1 virus alone, and were similar levels to live virus-infected cultures. These findings suggest that stimulation of TLR7/8 on B cells can enhance GC B cell response to inactivated H1N1 virus vaccines in PEG-4MAL-based PBMC organoids. Similar to the inactivated A/PR/8/34 H1N1 virus, organoids cultured with 10 μg/mL of Caledonia H1N1 antigen and 5 μM TLR7/8 agonist exhibited a higher number of GC B cells per organoid after 12 days of culture, following the order H1N1+R848>H1N1>R848 or media. Based on these findings, the study compared H1N1 with H1N1+R848 in the remainder of the experiments.

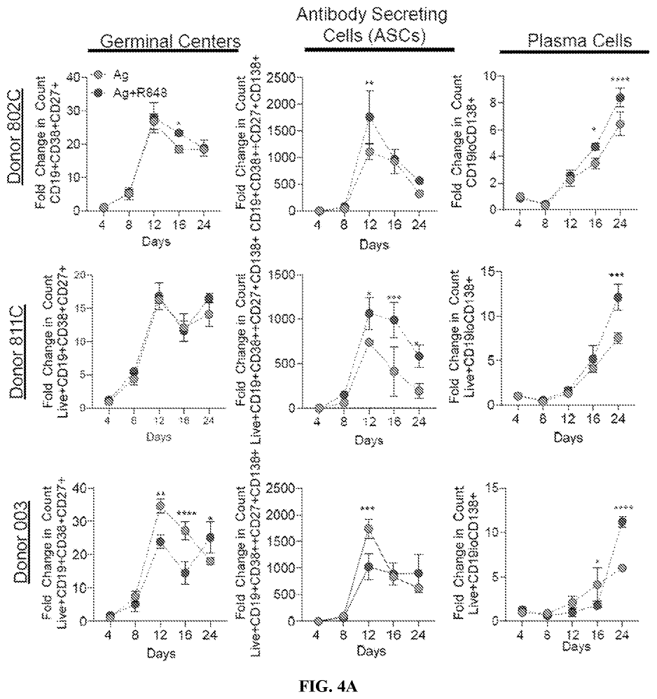

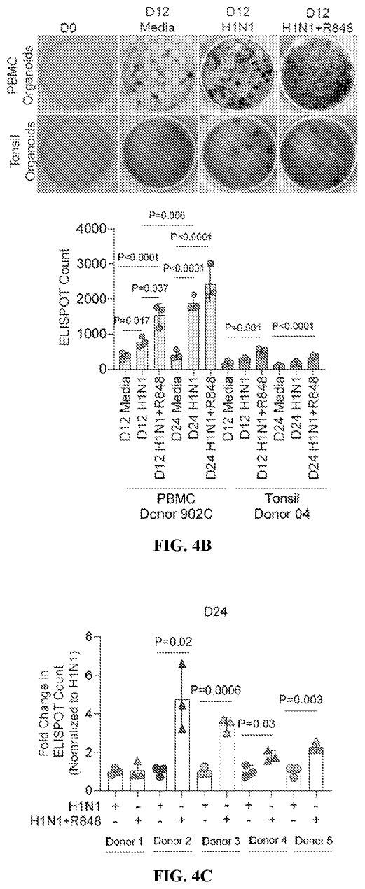

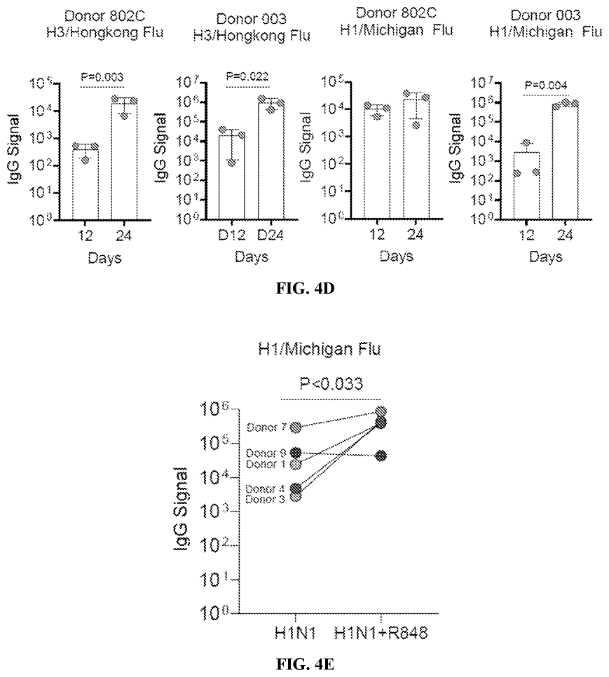

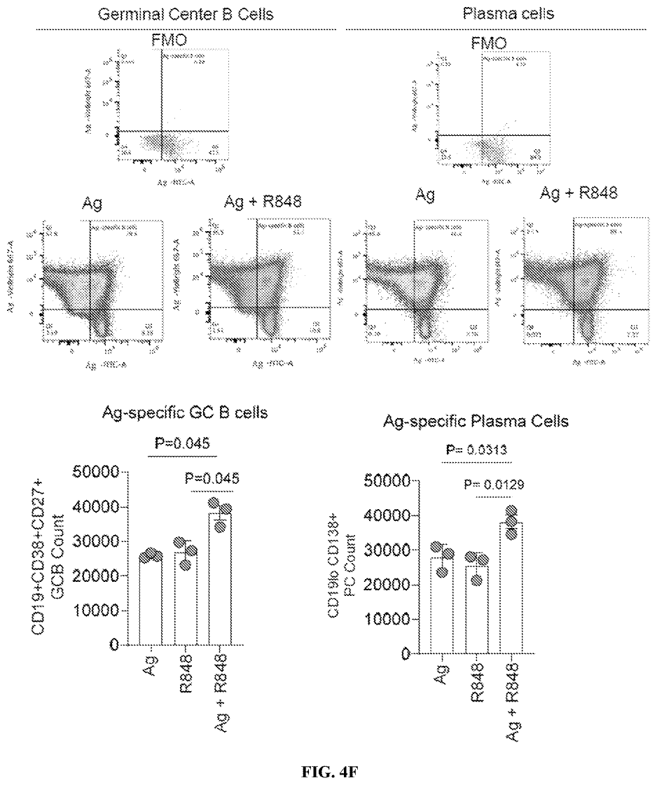

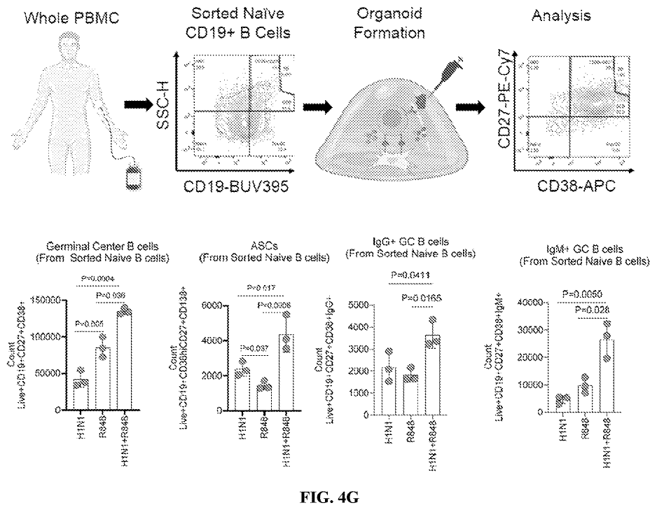

We next evaluated the response across multiple donors using inactivated New Caledonia 20/99 (H1N1) influenza A virus, with and without TLR7/8 agonist. We observed donor-dependent responses in organoids developed using 4 PBMC donors (FIG. 4A). While all donors demonstrated a prolonged 24-day response, H1N1+R848 induced significantly more GC B cells in 2 out of 4 donors after 12 days, and the opposite response in the remaining two donors (FIG. 4A). Treatment with H1N1+R848 induced more plasma cell response in 3 donors after 24 days (FIG. 4A) and no differences in the fourth donor. The donor variability was also observed with 5 tonsil donors, with most tonsil organoids declining their output after day 12-16.

To understand the differences between inactivated H1N1 antigen versus H1N1+R848 combination in PBMC and tonsil organoids, the study measured several inflammatory cytokines from organoid culture supernatants on early (day 4) and late (day 12) days. As critical antiviral mediators that are central to the elimination of viruses, IFNγ, IL6, and TNFα were measured using a Meso Scale Discovery U-Plex platform. The addition of R848 increased these inflammatory cytokines compared to the H1N1 vaccine alone. The cytokine levels mostly dropped at 12 days and could be attributed to culture conditions where the vaccine was only added for the first 2 days and the media was changed every 2 days. An important signal for an effective GC response, IL10, has been reported to promote humoral immunity by limiting excessive IFNγ [52]. IL10 demonstrated similar changes from day 4 to day 12 as IFNγ. Although the MesoScale assay was able to measure the soluble cytokine in the medium, the study was also interested in understanding the cytokine expression in B cells. RNA-FISH imaging of hydrogel-based organoids was used to visualize cytokine mRNA expression at the sub-cellular level. With this technique, the study validated that H1N1+R848 exposed CD20-expressing B cells had more expression of IL10, TNFα, IFNγ, and IL6 than H1N1 alone.

### Example 6: Immunostimulation Using TLR7/8 Agonist Induces Light Zone and Dark Zone Compartmentalization Against H1N1 Vaccines

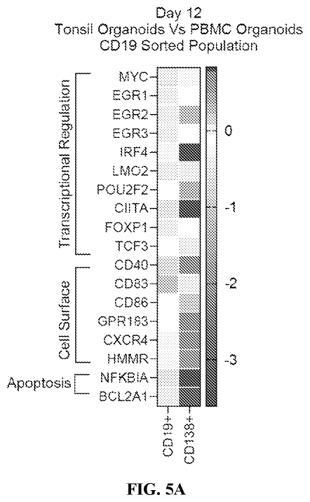

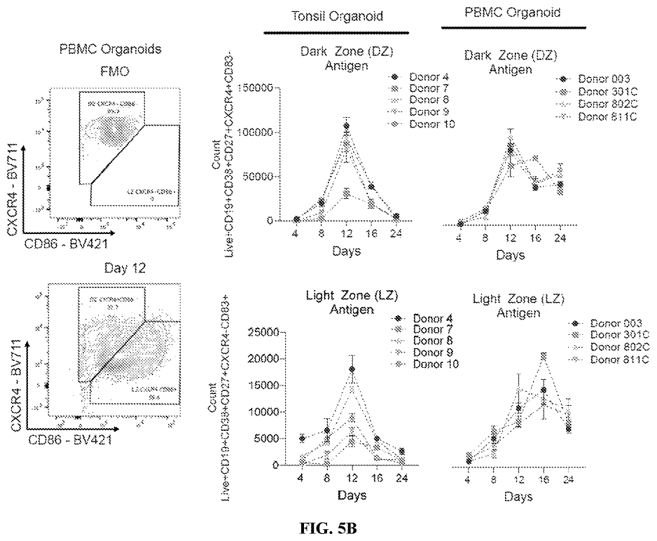

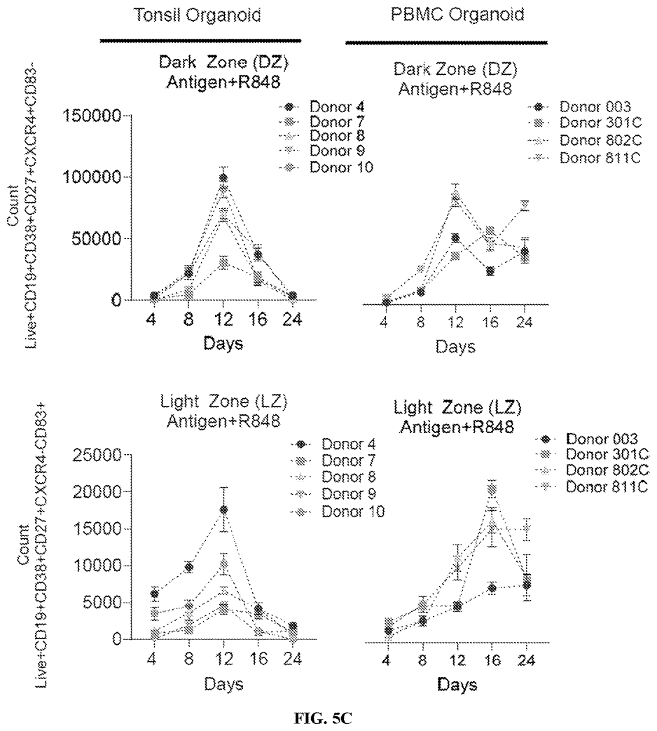

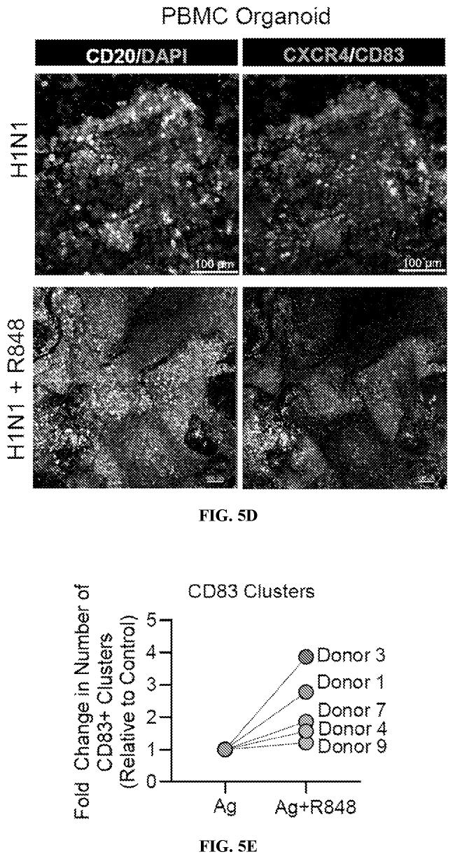

To undergo somatic hypermutation and affinity maturation, and to develop into ASCs, GCs undergo dynamic rearrangement between dark zones and light zones, mediated by chemokines and corresponding receptors on B cells [11]. Dark zone and light zone polarization is regulated by the expression of the stromal chemokines CXCL13, which signals through CXCR5 in the light zone, and CXCL12, which signals through CXCR4 in the dark zone [9]. Centroblasts expressing a CXCR4hiCD83lo or CXCR4hiCD86lo profile represent a proliferative (optically dense) dark zone whereas CXCR4loCD83 hi or CXCR4loCD86 hi centrocytes are localized in the light zone [9] [58]. The orchestration and spatial separation of dark zones and light zones are crucial for B cell maturation. Interestingly, bulk RNA-sequencing analysis of sorted CD19+ and CD138 B cells on day 12 showed that PBMC-derived organoids upregulated CXCR4 in CD19 population and downregulated in CD138+ population (FIG. 5A). In contrast, CD83 and CD86 was more expressed in tonsil-derived CD19+ cells than in PBMC-derived cells, and CD138+ plasma cells showed opposite response. These findings prompted the exploration of the temporal differentiation of centroblasts and centrocytes over 24-day organoids culture. In tonsil-derived organoids, CXCR4+CD83− dark zone GC B cells peaked on day 12 and declined afterward with few cells remaining by day 24. In contrast, for PBMC organoids, dark zone populations peaked at day 12 and remained elevated for most donors between 12 and 24 days (FIG. 5B). Interestingly, the peak of CXCR4-CD83+ light zone GC B cells was seen on day 16 in PBMC-derived organoids, as compared to day 12 in tonsil-derived organoids. The number of light zone GC B cells did not reduce significantly for most PBMC donors over 24 days, which was in stark contrast to tonsil organoids (FIG. 5B). Additionally, the number of dark zones and light zones GC B cells per PBMC organoid was comparable between H1N1 alone and H1N1+R848 treatment (FIG. 5C), with modest improvement with the adjuvanted condition. The variable differentiation dynamics over 24-day culture from all donors suggests that the platform technology can capture donor heterogeneity with both tonsil organoids and PBMC organoids. The t-SNE clustering analysis of light and dark zone markers suggested that these markers were largely co-localized with the GC B cell population and not memory or plasma phenotypes.

To visualize light and dark zone spatial organization in the organoids, organoids were stained with CD83, CXCR4, and CD20 antibodies. With H1N1 alone treatment, it was observed that the light and dark zone GC B cells were distributed randomly in the tonsil organoids. However, with the addition of R848 in the tonsil organoids, the light and dark zones separated into two distinct zones. In contrast, PBMC organoids that were treated with H1N1+R848 formed multiple distinct light and dark zones (FIG. 5D). Through cluster quantification (see methods) with additional staining from 5 donors, it was confirmed that H1N1+R848 leads to a higher formation of light zone CXCR4-CD83+ clusters compared to treatment with H1N1 alone (FIG. 5E). These findings suggest that although the quantitative flow cytometry shows a comparable number of cells across tonsil and PBMC organoids, or across treatment groups, the confocal imaging demonstrates a more distinct spatial impact of treatment groups.

### Example 7: Hydrogels Presenting Lymphoid ECM, Stiffness, Cellular Signals Regulates Ex Vivo B Cell Survival

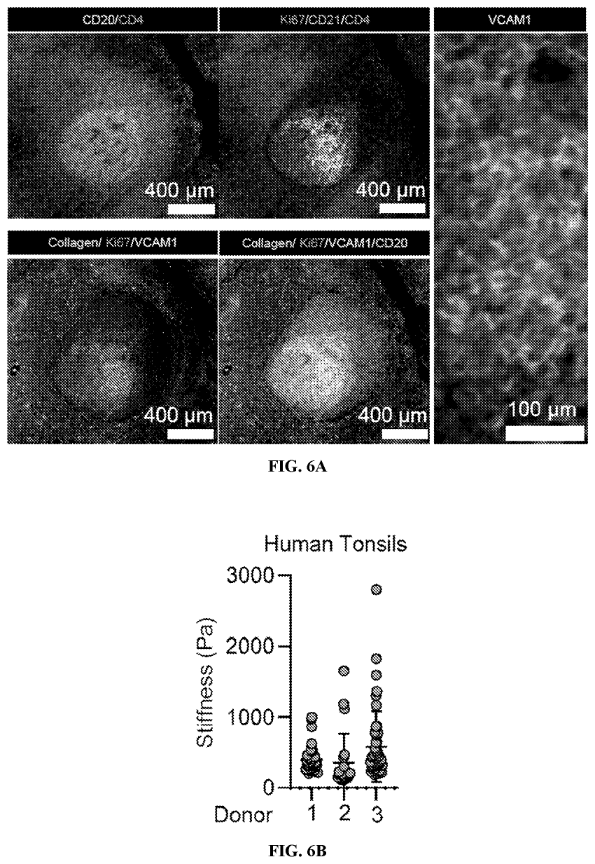

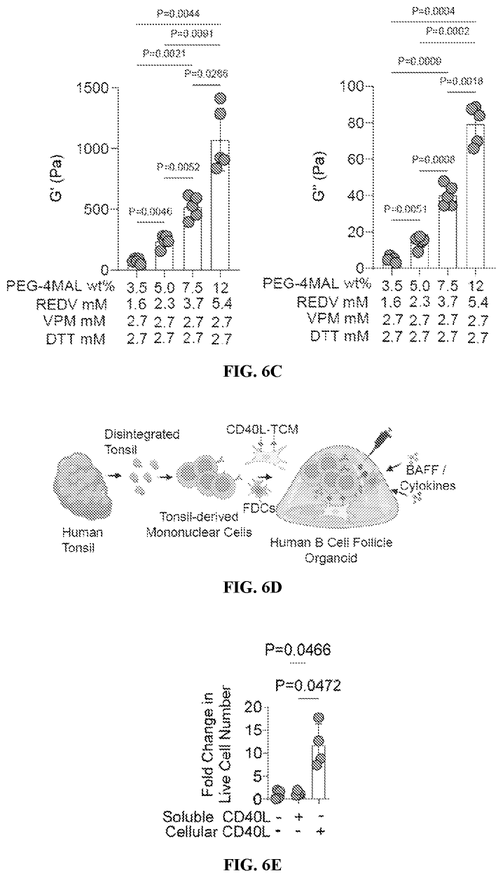

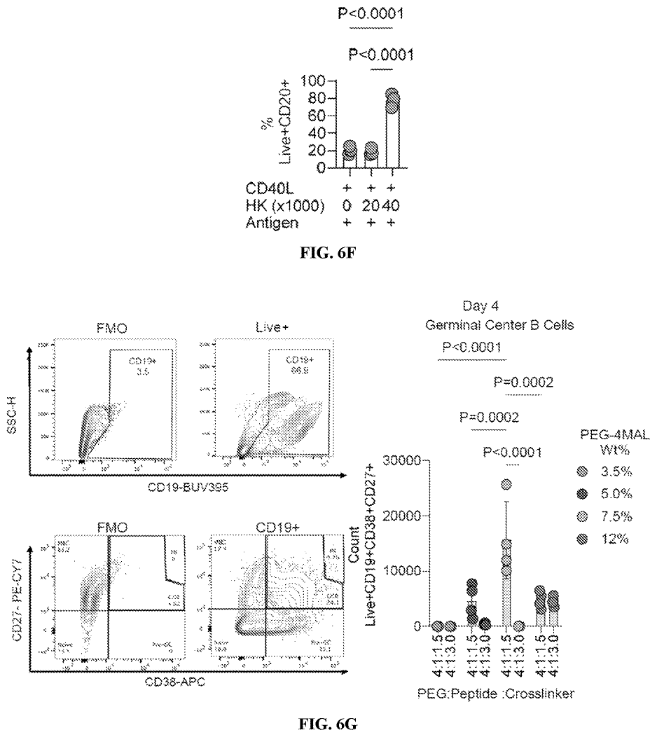

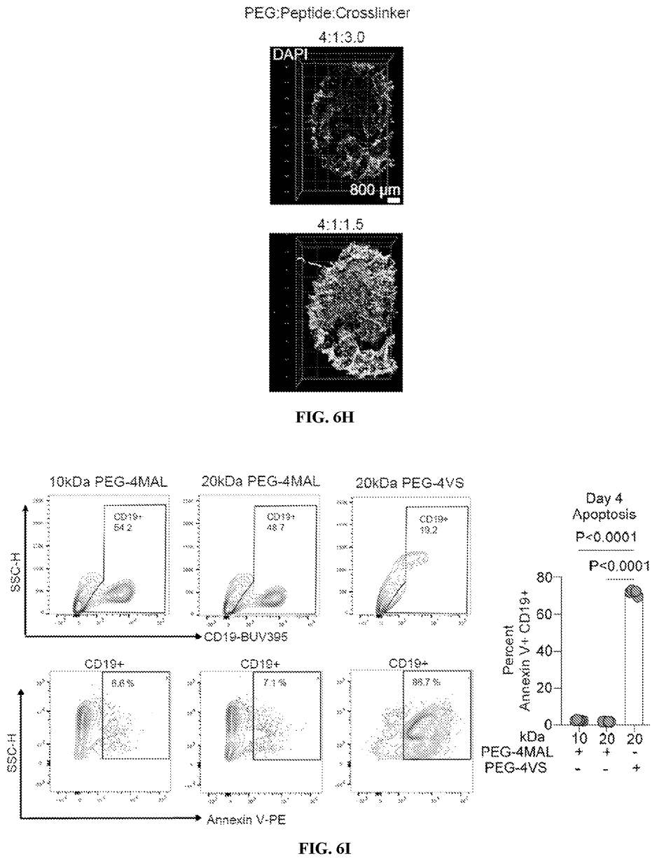

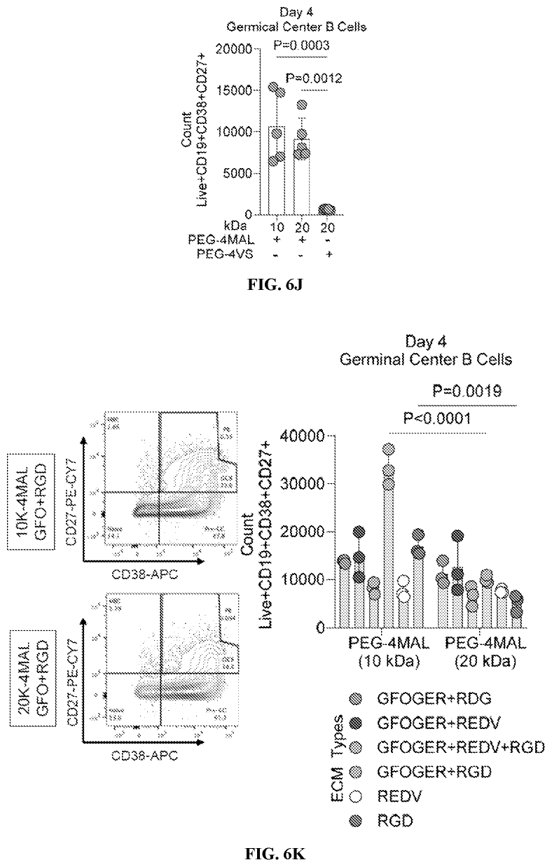

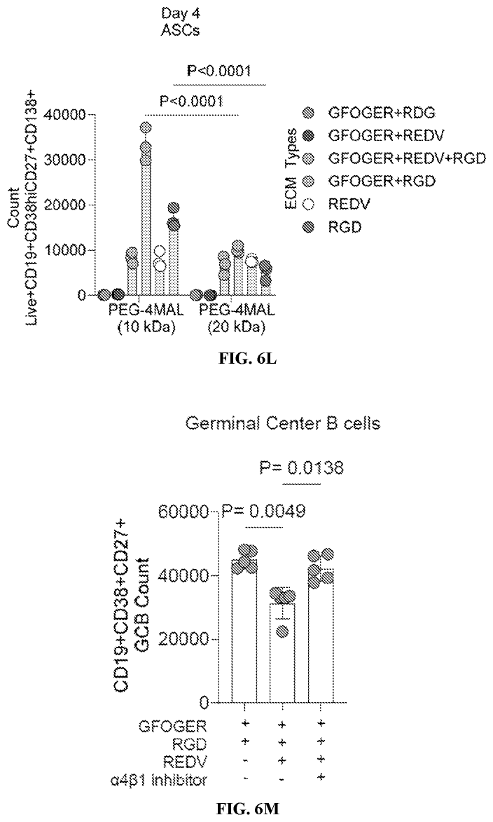

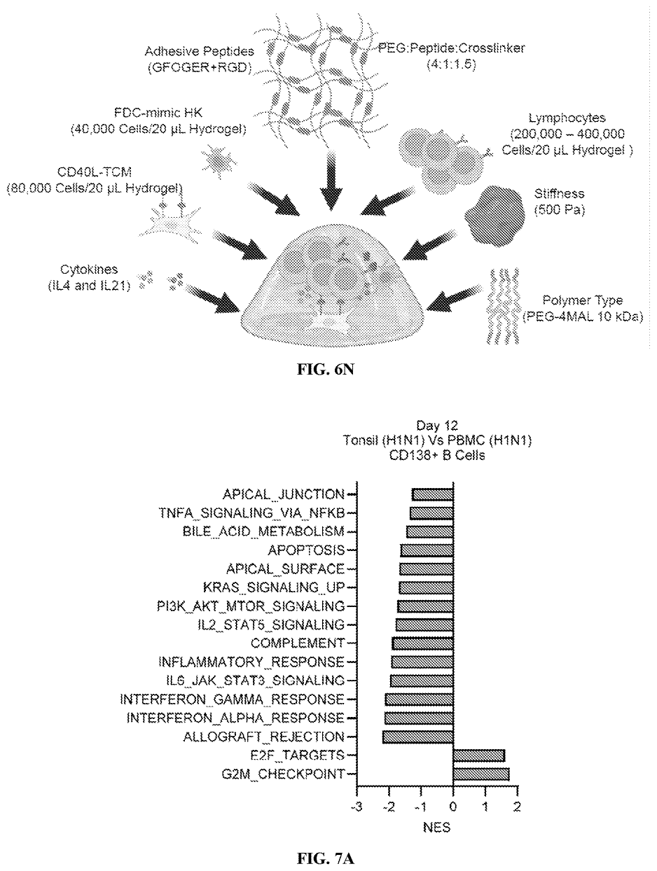

Immune cells in secondary lymphoid organs, such as tonsil and lymph nodes, dynamically interact with the local three-dimensional microenvironment through cell-cell, cell-extracellular matrix (ECM), and biophysical interactions with tissues. During B cell differentiation, naïve B cells interact with stromal cells, such as follicular dendritic cells (FDCs), and follicular T helper cells (TFH) through multiple receptor-ligand interactions and secreted cytokines and chemokines [21] [22]. Whereas the antigen can directly bind to B cells or can be presented through FDCs [23], integrin α4β1 (very late antigen-4) expression plays a key role in stabilizing the initial interaction of B cells with antigens associated on the surface of FDCs that express its ligand, vascular cell adhesion protein-1 (VCAM-1), which in turn enables B cell activation [24]. To bioengineer synthetic immune organoids that mimic key cellular and ECM components of tonsil lymphoid tissues, a study performed cyclic immunofluorescence staining on tonsil tissue sections. The study observed B cells (CD20+), including proliferative B cells (CD20+Ki67+), were present surrounded by ECM (collagen I+, VCAM1+), T cells (CD4+), and FDCs (CD21+) (FIG. 6A). Imaging mass cytometry further revealed that B cells were located in the vicinity of TFH (PD1+CD4+CXCR5) cells and CD21+FDCs.

The mechanical properties of secondary lymphoid tissue [25] [26] [27] influences the structure and function of the immune cells [28] [29]. To bioengineer synthetic immune organoids that mimic the mechanical properties of natural tonsil tissue, the study first quantified the stiffness of freshly isolated tonsil tissues from three independent human tonsil donors using atomic force microscopy (FIG. 6B). The average tonsil stiffness was ~500 Pa, with no significant differences between the donors.

To incorporate the ECM, cellular, and mechanical stiffness observations from the natural tonsils, the study engineered a synthetic hydrogel to first present VCAM-1-mimicking peptides and modulated the resulting tissue's biophysical properties. A 4-arm poly(ethylene glycol) macromer end-functionalized with maleimide groups (PEG-4MAL) was conjugated to cysteine-flanked, VCAM1-mimicking REDV peptides (3.7 mM final density to maximize integrin-mediated adhesion sites [30] [31] [32]). These macromers were crosslinked into a hydrogel with a mixture of protease degradable crosslinker peptide (GCRDVPM SMRGGDRCG, referred to as VPM) and non-degradable crosslinker dithiothreitol (DTT), previously optimized to provide stability to B cell cultures [32](FIG. 6C). To engineer a hydrogel that mimicked the tissue stiffness of tonsils, the study evaluated the storage modulus for hydrogels that presented fixed VPM and DTT concentration, with increasing PEG-4MAL wt %. The resulting storage modulus for 7.5% hydrogels was 518±42 Pa, whereas that at 12% was 1075±76 Pa (FIG. 6C). The 3.5 and 5 wt % gels were much softer, with storage modulus in the 75±9 Pa and 239±22 Pa, respectively (FIG. 6C). The loss moduli of the gels followed a similar trend, and the G′/G″ indicated that the gels behaved as elastic solids (FIG. 6C). These rheological results at 7.5% were comparable to those obtained from hydrogels fabricated with fixed adhesive peptide densities with increasing PEG-4MAL wt %. Rheological testing indicated that a 7.5 wt % PEG-4MAL hydrogels achieved a stiffness that paralleled that of natural tonsil tissue.

In vivo, the presence of antigen activates CD4+ T cells to differentiate into follicular T helper cells that express CD40L, which engages with CD40 on B cells to initiate GC reactions [33]. To test whether the T-cell signal CD40L could be presented as a soluble recombinant protein for B cell activation, the study compared soluble CD40L [34] [35](100 ng/mL) to CD40L-expressed on an engineered fibroblast line (a T cell mimic (CD40L-TCM) that was recently reported [32]. A mononuclear fraction of 200,000 freshly isolated tonsil single cells were encapsulated with soluble CD40L or CD40L-TCMs in a 20 μL of 7.5 wt % PEG-4MAL (10 kDa) hydrogels (FIG. 6D), based on recent work with human B cell lymphomas [32]. The hydrogels were casted in a 96-well plate and in the initial set of optimization studies, human IL-4 (20 ng/mL) with B cell activating factor (BAFF) (100 ng/mL) was added for 4 days. Flow cytometry analysis indicated that presentation of CD40L in its membrane-bound format as CD40L-TCM increased the viability of total tonsil mononuclear cells by 11.7±2.3 fold compared to no CD40L conditions, while soluble CD40L condition increased viability by only 1.2±0.3 fold (FIG. 6E). We next investigated the role of human FDCs on B cell expansion. FDCs are a specialized type of antigen-presenting dendritic cell found in B cell follicles, and their primary function includes maintaining the follicular architecture, binding and retaining antigens through immune complexes, secreting chemokines, and presenting these antigens to B cells for GC reaction. FDCs also rescue antigen-bound B cells from apoptosis [22]. Therefore, the study incorporated human tonsil-derived FDC, called HK-FDCs [36] [37], and observed that the percentage of live tonsil mononuclear cells increased in an HK-FDC density-dependent manner, with a high 77.9±4.5% live B cells obtained with 40,000 HK-FDCs per hydrogel, as compared to 18.5±2.2% live B cells with 20,000 HK-FDCs per hydrogel (FIG. 6F). Notably, both CD40L and HK-FDC presence were needed to sustain the survival of CD20+ B cells. We also tested whether encapsulated cells would impact the mechanical properties of the gels over time, and indeed the storage modulus was reduced to ~50-60% over 4 days but remained the same up to 12 days of culture.

GC B cells in follicles are diffused aggregates of specialized B cells that undergo rapid proliferation. To generate a polymer network of PEG-4MAL that allowed for high cell proliferation and aggregation, the study varied polymer density and crosslinker density and determined the effect on mechanical properties of the hydrogel and fate of B cells. We tested two crosslinking densities by using (PEG-4MAL):(adhesive peptide):(total crosslinker) molar ratios of maleimide to thiols, designated them as 4:1:1.5 and 4:1:3.0, and varied weight % of PEG-4MAL. The REDV concentration was kept fixed at a particular PEG-4MAL wt % for both 4:1:1.5 and 4:1:3.0 but varied with change in PEG-4MAL wt %. The rationale for this approach is that with 4:1:1.5 composition hydrogel, it was assumed that REDV would fully occupy 1 arm of PEG-4MAL when the mixture of PEG-4MAL was prepared prior to crosslinking, and all the remaining 3 arms of PEG-4MAL is theoretically occupied by crosslinkers. For reference, at 7.5 wt %, 4:1:1.5 refers to concentration of PEG-4MAL=7.5 mM, adhesive peptide REDV=3.75 mM, and crosslinker VPM=2.7 mM, DTT=2.7 mM, whereas 4:1:3 refers to MAL=7.5 mM, REDV=3.75 mM, VPM=6.2 mM, DTT=6.2 mM. The 4:1:1.5 hydrogel composition at 7.5 wt % led to a significantly higher number of GC B cells (Live+CD19+CD27+CD38+) and antibody-secreting cells (ASCs: Live+CD19+CD38hiCD27+CD138+) as assessed by flow cytometry on single cells recovered from protease-digested hydrogels at day 4 (FIG. 6G). The high cell numbers in 4:1:1.5 hydrogel composition was further supported by confocal imaging (FIG. 6H). These initial findings confirmed the optimal formulation is a 7.5% PEG-4MAL hydrogel with 4:1:1.5 (PEG-4MAL):(adhesive peptide):(total crosslinker) molar ratios, with ~500 Pa storage modulus.

### Example 8: PEG Macromer Size and Type Regulate Primary Human B Cell Survival

In the quest to define the most suitable biomaterial with optimal physiochemical properties that allow for B cell survival and differentiation, the study next examined B cell viability in hydrogels generated from different macromers: 10 kDa PEG-4MAL, 20 kDa PEG-4MAL, and 20 kDa 4-arm PEG-vinyl sulfone (PEG-4VS) using the same concentration of REDV peptide and crosslinking density. PEG-4VS, with less efficient Michael-type addition gelation than PEG-4MAL [31], has previously been reported for the development of bioadhesive, protease-degradable hydrogels [38] [39]. The storage modulus of 7.5 wt % 10 kDa PEG-4MAL was 544±6 Pa and 7.5 wt % 20 kDa PEG-4MAL and PEG-4VS hydrogel formulations were 65±5 Pa and 55±7 Pa, respectively. Despite similar storage moduli between 20 kDa PEG-4MAL and 20 kDa PEG-4VS, it was observed that PEG-4VS gels showed a 20-fold higher percentage of apoptotic B cells (72±0.6%) than those of the other two PEG-4MAL macromers (FIG. 6I). Additionally, PEG-4VS gels displayed a 15-fold lower number of GC B cells (Live+CD19+CD38+CD27+) than the other two hydrogel formulations over 4 days of culture (FIG. 6J). A few plausible explanations for why the B cells appear sensitive to formulations of the matrix include, but not limited to 1) rapid reaction kinetics of Maleimide bonds with thiolated crosslinkers [31], preventing any toxic exposure to unreacted groups, and 2) the increased reversibility of bond-forming reaction in Maleimides than that of vinyl sulfones [40]. In Maleimide-thiol adducts, the Michael-type addition of thiols to N-ethylmaleimide typically results in the formation of a stable succinimide thioether; however, this adduct can undergo retro and exchange reactions in the presence of other thiol compounds at physiological pH and temperature, which may create a more favorable environment for B cell survival. More fundamental studies are needed to understand the differences between the impact of formulations on B cells.

### Example 9: Combination of Collagen and RGD Peptides in Hydrogel Support B Cell Maturation

Whereas VCAM-1 is an important cell-based signal and integrin α4β1 is expressed on B cells, prior studies have shown enrichment of RGD-containing ligands (vitronectin) in the GC upon immunization, leading to improved plasma cell responses [41], and imaging of human tonsil tissues indicated the presence of collagen type I around B cell follicles (FIG. 6A), with partially interspersed collagen within the follicles around the proliferative B cells (Ki67+). To understand the effect of integrin-binding ligands on human GC B cell induction in the organoids, the study functionalized PEG-4MAL hydrogels with different bioadhesive peptide combinations: ‘REDV’, fibronectin mimic ‘RGD’ (GRGDSPC), and collagen-I-mimetic peptide ‘GFOGER’ (GYGGGP(GPP)5GFOGER(GPP)5GPC, where O=hydroxyproline). GFOGER binds to multiple collagen-binding 01 integrin dimers including α2β1 [32] [42]. Specific combinations included GFOGER+RGD, GFOGER+RDG (a scrambled peptide), GFOGER+REDV, GFOGER+REDV+RGD, REDV, and RGD, all used at a final 3.7 mM peptide density.

We observed that the combination of peptide ligand combinations and PEG-4MAL molecular weight had a distinct impact on GC phenotype development. Notably, the combination of RGD and GFOGER led to a 2-fold increase of GC B cells and ASCs compared with RGD alone or GFOGER alone in stiffer 7.5 wt % 10 kDa PEG-4MAL hydrogels, as compared to softer 7.5 wt % 20 kDa PEG-4MAL hydrogels (FIGS. 6K-6L). The combinatorial effect was significantly higher than that induced in hydrogels functionalized with RGD, REDV, or GFOGER alone. To confirm whether REDV was indeed interacting with B cells and partly inhibiting GC reaction, the study inhibited the interaction of REDV with integrin α4β1 using a commercially available α4β1 inhibitor. We observed that inhibiting the α4β1 integrin resulted in the recovery of GC B cells comparable to the GFOGER+RGD group, indicating that REDV binding to α4β1 may have a suppressive effect on GC B cell response (FIG. 6M). Although the cause behind it is not fully understood, it is plausible that REDV-α4β1 interaction in organoids induces suppressive cytokines that may reduce the GC response [43] [44].

Collectively, the characterization of tonsil microenvironment and rigorous testing of mechanical properties, ECM components, and molecular as well as cellular signals, resulted in an optimized composition of synthetic immune organoids (FIG. 6N) that maximized the B cell survival and differentiation into GC phenotype and ASCs. The final optimized conditions were a 10 kDa, 7.5% PEG-4MAL at 4:1:1.5 molar ratio, 500 Pa stiffness with a combination of GFOGER+RGD peptides, and was used in the remainder of studies.

### Example 10: Cellular Composition and Tissue Source Regulates B Cell Maturation in Organoids

The differences seen in PBMC versus tonsil organoids raise the question of presence of distinct cellular types in tonsils than PBMCs and the B cells themselves being harvested from a tissue versus peripheral blood. TFH cells are a specialized subset of T cells that play a crucial role in the development of long-lasting and highly effective antibody responses. Using flow cytometry, a study measured the presence of TFH in both tonsil-derived and PBMC-derived organoids. While the tonsil-derived organoids initially had a higher number of TFH cells on day 0 (22% tonsil-derived versus 4% PBMC-derived), these numbers were comparable on day 4 between two types of organoids. Interestingly, PBMC-derived organoids displayed a 3-fold increase in TFH cell numbers between day 4 and day 12. In contrast, the tonsil-derived organoids maintained a steady level of TFH cells without a significant increase (FIG. 3M). TFH also interacted with B cells in the organoids (FIG. 3N). Flow cytometry confirmed higher proportion of naïve B cell in PBMCs but tonsil had more over all CD19+ B cells, pre-GC and GC B cells. Both PBMC and tonsil populations had comparable memory B cells.

We performed bulk RNA-sequencing on CD19+ B cells from tonsil and PBMC samples on day 0 and found differentially expressed gene sets (DEGs) with over 600 DEGs with increased expression and approximately 1000 with reduced expression, suggesting an already distinct state of tonsil-derived B cells than PBMC-derived B cells. GSEA pathway analysis on CD19+ B cells from each source further revealed upregulation of inflammatory signaling (Inflammatory response, TNFα signaling, IL6_JAK_STAT3 signaling, etc.) and proliferation (KRAS signaling) in tonsil-derived B cells. Conversely, PBMC-derived CD19+ B cells show upregulation of pathways essential for their survival (PI3K_AKT_MTOR), metabolic regulation (Fatty acid metabolism, Glycolysis, etc), and the control of cell division (MYC, G2 μM checkpoint, E2F target, etc) (FIG. 3P).

To discern the causes behind the distinct B cell differentiation dynamics in tonsil-derived versus PBMC-derived organoids, immune organoids were fabricated using FACS-purified naïve B cells from both sources. Contrary to an anticipated decline post-day 12, it was found that that the naïve B cells from tonsil tissue not only persisted but also continued their differentiation into plasma cells up to day 24. However, those from PBMCs exhibited a higher fold change of differentiation (FIG. 3O). This result indicates that the presence of non-naïve B cells in tonsil-derived organoids negatively influenced B cell maturation dynamics.

### Example 11: Organoids Demonstrate GC Formation in Purified Naïve B Cells and Generate Antigen-Specific Response

We next investigated the generation of antigen-specific B cells on Day 12 with ELISPOT. Compared with tonsil organoids, PBMC organoids demonstrated more antigen-specific responses on Day 12, with ELISPOT spots in H1N1+R848 being significantly higher than those in H1N1 alone or media groups on both Day 12 and 24 (FIG. 4B). In contrast, 2D co-cultures formed fewer spots. We noted the same trend of increase in total CD19+ B cells within PEG-4MAL based organoids subjected to H1N1 with the addition of R848 compared to H1N1 alone. This pattern of increase, however, was not replicated in 2D co-cultures. The ELISPOT results were dependent on donors as 4/5 new PBMC donors responded to addition of R848 to H1N1 (FIG. 4C). We performed an electro-chemiluminescent-based multiplex immunoassay provided by Mesoscale Discovery (MSD-ELICA) on supernatant media and observed significantly higher flu-specific IgG titers in organoids treated with H1N1+R848 than H1N1 alone (FIG. 4D), further supporting ELISPOT results. Notably, the secreted flu-specific IgG antibody pattern demonstrated patient heterogeneity, with two tested donors responding distinctly to H3/Hong Kong flu and H1/Michigan flu. Similar to the ELISPOT findings, the H1 antigen-specific IgG results were dependent on randomly chosen additional 5 donors and 4/5 of these PBMC donors responded to addition of R848 to H1N1 (FIG. 4E). To further validate these findings with flow cytometry, a study examined H1N1-specific GC B cells and plasma cells using a combination of H1N1-tetramer of biotin-conjugated H1N1 with streptavidin-FITC and streptavidin-VioBright667. We found that the H1N1+R848 significantly enhanced antigen-specific GC B cell and plasma cell populations (FIG. 4F).

Lastly, given the widespread H1N1 infections and vaccinations, a PBMC sample can have both naïve and memory B cells against H1N1 [53], and memory B cells can respond to R848 stimulation alone. We removed memory B cells by using a naïve B cell sorting kit and incubated naïve (CD3-CD19+CD27−) B cells with CD40L-TCM and FDCs as before, along with BAFF, IL4, and IL21 cytokines. The study observed an increase in GC B cells and ASCs in organoids exposed to H1N1+R848 compared to H1N1 alone or R848 alone (FIG. 4E). Strikingly, exposure to H1N1+R848 induced expansion of IgM+ B cells, but at the same time induced significantly higher class-switching to IgG+ GC B cells (FIG. 4E). Based on these findings, in further studies focused on comparisons between H1N1 and H1N1+R848.

### Example 12: Differentially Expressed Gene Sets in PBMC Organoids Exposed to H1N1 with TLR7/8 Agonist

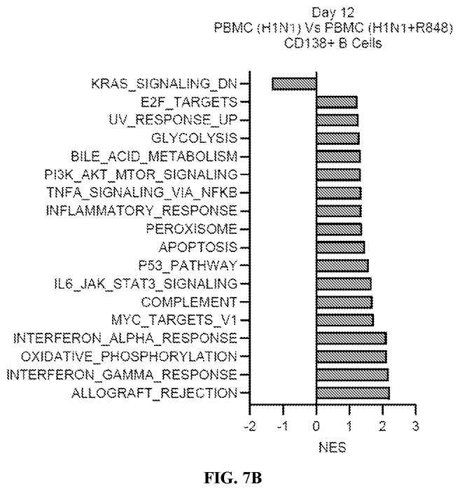

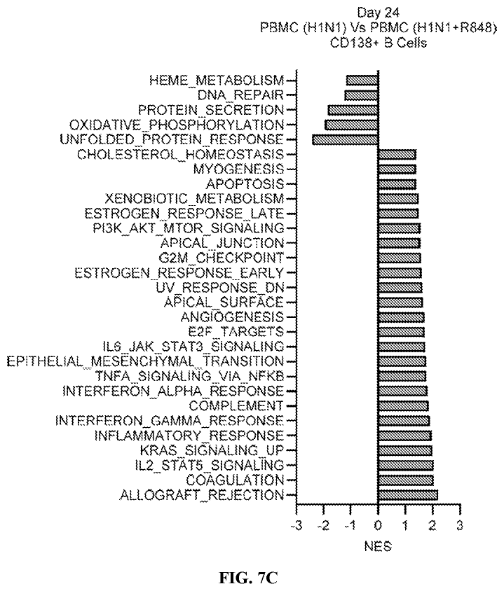

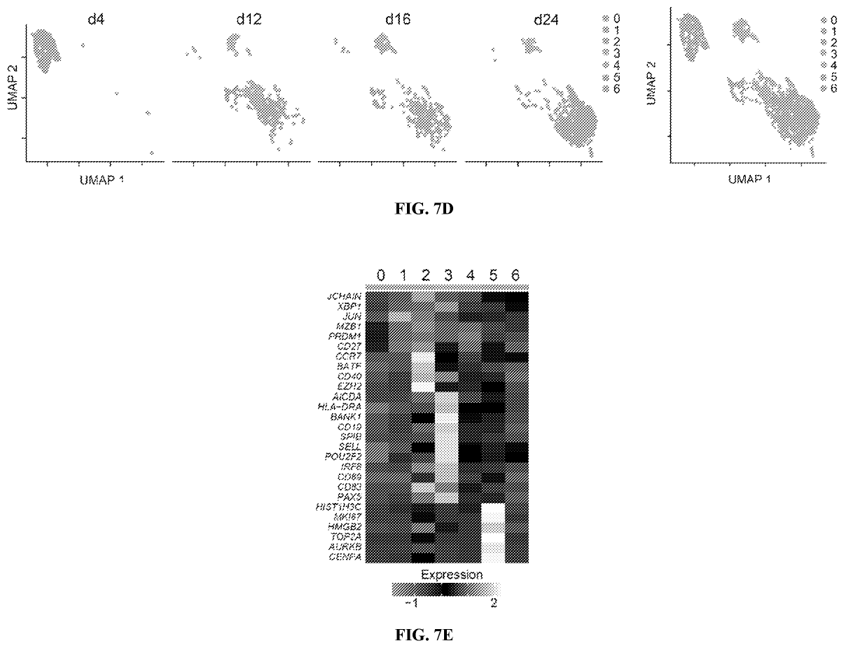

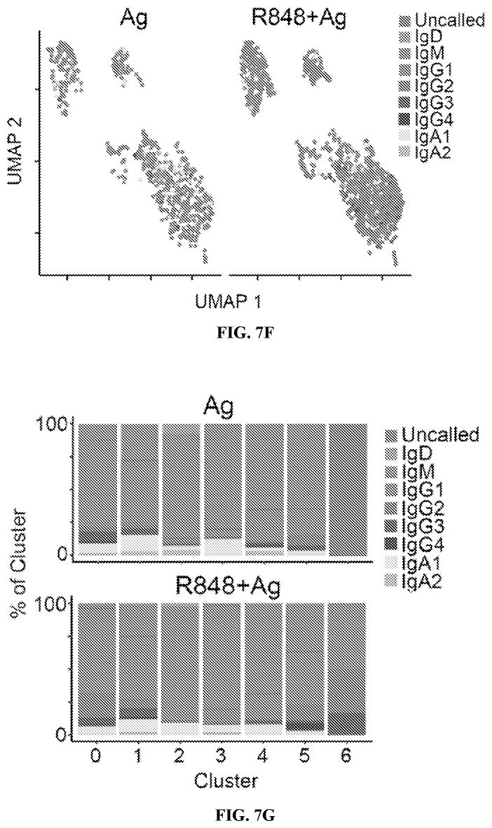

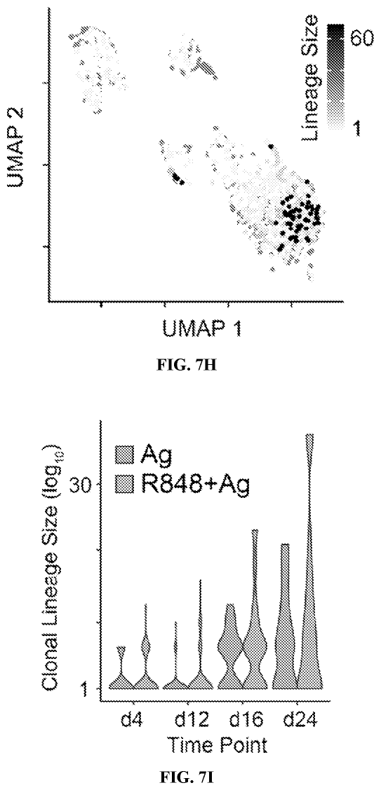

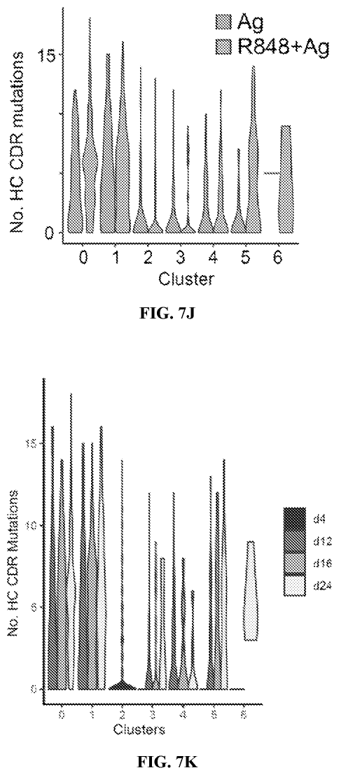

Since the ultimate goal is antibody secreting cells (ASCs), a study performed bulk RNA-Seq to discern the differences in CD138+ B cells across organoids. At day 12, the study observed 709 differentially expressed gene sets (DEGs) in H1N1 exposed organoids, and 334 DEGs in H1N1+R848 exposed organoids, comparing tonsil-derived and PBMC-derive organoids. In H1N1 exposed organoids, Gene set enrichment analysis (GSEA) indicated upregulation of multiple pathways in PBMC-derived CD138+ B cells in organoids than tonsil-based organoids, including those related to interferon and inflammatory responses, Stat3 and Stat5 signaling, PI3k-AKT-mTOR signaling, bile acid and TNF signaling via NFkB (FIG. 7A). Downregulated pathways included those related to E2F targets and G2 μM checkpoints (FIG. 7A). H1N1+R848 further downregulated glycolysis and MYC signaling in PBMCs.

We next performed bulk RNA-Seq on CD138+ B cells in PBMC-derived organoids on day 12 and day 24, when exposed to H1N1 versus H1N1+R848. Tonsil samples were not considered because of longevity issues through day 24. While the study found a modest number of DEGs of CD138+ B cells in PBMC-derived organoids on day 12 when comparing H1N1 versus H1N1+R848, this difference markedly increased to 2000 on day 24. Pathway analysis on day 12 indicated downregulation of multiple pathways in PBMC-derived CD138+ B cells in H1N1+R848 treated organoids, including those related to interferon and inflammatory responses, Stat3 and PI3k-AKT-mTOR signaling, bile acid and TNF signaling via NFkB, E2F targets, G2 μM checkpoints, glycolysis, oxidative phosphorylation, and MYC signaling (FIG. 7B). In contrast, pathway analysis on day 24 showed upregulated oxidative phosphorylation and DNA repair, while downregulating interferon and inflammatory responses, Stat3 and Stat5, PI3k-AKT-mTOR signaling, TNF signaling via NFkB, E2F targets, G2 μM checkpoints, and mTOR signaling (FIG. 7C).

TNF and TNF superfamily cytokine signaling play important roles in B cell and ASC survival and function [54]. The TNF suppression in tonsil-derived ASCs in organoids on day 12 (conversely, increased in PBMC-derived organoids), could explain improved performance of PBMC-derived ASCs. Factors BAFF (TNFSF13b) is essential for ASC survival and OX40 (TNFRSF4) are significantly upregulated in late stage ASCs [54]. While the study found TNFSF13b and upregulated in PBMC-derived CD138 B cells, both TNFSF13b and TNFRSF4 were upregulated on day 24 in PBMCs with more expression in Ag alone treated group than Ag+R848.

### Example 13: ScRNA Sequencing Reveals Temporal B Cell Maturation in PBMC-Based Organoids with TLR7 Agnonist

To understand the dynamics of B cell differentiation and the heterogeneity of B cell isotypes and clones during the organoid culture, a study performed scRNA sequencing, where B cells were sorted from organoid culture on Day 4, 12, 16, and 24. After exclusion of non-B contaminating cells (including B cells), low-quality cells, dying cells, and doublets, the remaining cells were identified as B cells and plasma cells based on Ig expression, BCR-seq data matching, and expression of key lineage genes. Utilizing uniform manifold approximation and projection (UMAP) [55] for dimensionality reduction, the study visualized the diversity of B cell subtypes within PBMC organoids, delineating six distinct clusters based on their gene expression profiles that emerged temporally over 24 days (FIGS. 7D-7E). Cluster 2, which reached its peak at day 4 (FIG. 7D), included genes indicative of early B cell activation (e.g., CD40, BATF, CD69, CCR7, HMGB2), B cell proliferation (e.g., EZH2, AICDA, AURKB), and differentiation towards a GC B cell phenotype (e.g., PAX5, SPIB, IRF8, CD83) (FIG. 7E). Cluster 3 primarily consisted of GC B cells, characterized by prolific proliferation, active somatic hypermutation, and plasma cell differentiation, as evidenced by high AICDA and PAX5 expression, alongside SPIB and POU2F2. As GCs mature into plasma cells, proliferative ASCs typically shed B cell markers such as MHC class II gene [54], HLA-DRA, which was suppressed in clusters 0, 1, 4, and 5. While proliferation factor OCT-2 (encoded by Pou2f2) [56] was highly expressed in GC B cell Cluster 3, it was downregulated in clusters 4, 5, and extinguished in 0 and 1. In contrast, XBP1, an essential transcription factor (TF) associated with the ASC unfolded protein response, was increased clusters 0, 1, 4, and 5, with highest in Cluster 1. PRDM1, another essential TF associated with plasma cells, was increased in clusters 0, 1, 4, and 5, with highest in Cluster 1 and 4. Mzb1, a cochaperone that is expressed during the terminal differentiation of B cells to ASC and is a key effector of Blimp1 in plasma cell differentiation, was also increased in clusters 0, 1, 4, and 5, with highest in Cluster 1 and 4. Human ASC also express high levels of CD27 [57], which were high in Cluster 1 and 4. Cluster 5 distinctly expressed MKI67, consistent with the definition of proliferative plasmablasts [54] with TFs (XBP1, PRDM1), MZB1, and genes involved in somatic hypermutation, class switching, and clonal expansion (e.g., HIST1H3C, TOP2A). Nascent ASCs mature into ASCs demarcated by downregulation of CD19. CD19 expression was notable in Cluster 2 and 3, with low expression in Cluster 4, 5, and extinguished in late clusters 0, 1 suggesting maturation into plasma cells. Furthermore, the study confirmed that ASC underwent transitional phases (Cluster 4 and 5) and evolved into more mature forms (Clusters 0 and 1) between day 12 and day 24 (FIGS. 7D-7E). Collectively, these findings demonstrate that PBMC-organoid derived ASCs and plasma cells manifest many similar characteristic expressions of XBP1, IRF4, PRDM1, CD27, CD138, and CD19, as reported in bona fide ASCs found in human bone marrow [54]. Cluster 6 was an intermediate, undefined cell state that aligned more with ASCs than GC B cells.

### Example 14: BCR Sequencing Reveals Class Switching and Clonal Expansion of CD138+ B Cells

Immunoglobulin (Ig) class switching analysis using BCR sequencing, comparing Ag and Ag+R848, revealed a predominance class switching to IgG1 BCR Clusters 0 and 1, where Ag+R848 showed more IgG1 than Ag (FIGS. 7F-7G). We noted a progressive decrease in IgM and an increase in IgG1 expression from Cluster 2 through Cluster 5, with high IgM expression in Cluster 2. Compared to Ag alone, Ag+R848 increased IgG1 and IgG2 levels in Clusters 0 and 5. In contrast, Ag alone showed predominant IgG whereas Ag+R848 induced class switching to IgG1, IgG2, and IgG4 in Cluster 6. These findings demonstrate robust Ig class-switching, demonstrated through BCR sequencing. Furthermore, Cluster 0, composed of plasma cells, showed the largest clonal expansion of B cells (FIG. 7H). Ag+R848 induced more clonal expansion than Ag alone across all time points and almost all clusters (FIG. 7I).

Antibodies accumulate mutations in their complementarity-determining regions (CDRs) during affinity maturation. Somatic hypermutation (SHM) provides important information regarding the maturation of ASCs and may offer important insight into their differentiation from separate B cell sources. We assessed the mutation levels within the heavy chain CDR mutations and PBMC-derived B cells in all clusters showed evidence of SHM (FIG. 7J). ASCs and plasma cells in Clusters 0, 1, 4, and 5 had a higher number of heavy chain CDR mutations relative to activated B cells and GC B cells in Cluster 2 and 3 (FIG. 7J). In general, within the ASC and plasma cell clusters, the number of heavy chain CDR mutations in Ag+R848 conditions were higher than those in Ag alone conditions. Corroborating with the Ig class switching, Cluster 6, which showed more diverse Ig class switch under Ag+R848 conditions than Ag alone, also showed heavy chain CDR under Ag+R848 conditions. Lastly, the mutations in the CDR region continuously increased over 24 days in Cluster 0, 1, 3, and 5, with the highest on day 24 (FIG. 7K). Overall, the organoid-derived ASCs and plasma cells displayed SHM numbers that increased over time and accumulated in more mature cells. This observation corroborates with prior reports that show higher CDR mutations in bone marrow ASCs than naïve B cells [54]. In addition, comparing lower HLA-DR expressing Cluster 1 with Cluster 4, the heavy chain CDR in Cluster 1 was higher than in Cluster 4. This corroborates with previously reported findings that early-stage ASCs with MHC class II gene expression had a lower mutation frequency than late-stage ASCs [54].

The following patents, applications and publications as listed below and throughout this document are hereby incorporated by reference in their entirety herein.

### REFERENCE LIST

1. Chang, A. et al. Humoral Responses Against SARS-CoV-2 and Variants of Concern After mRNA Vaccines in Patients With Non-Hodgkin Lymphoma and Chronic Lymphocytic Leukemia. J Clin Oncol 40, 3020-3031 (2022).
2. Quinn, M. et al. Persons with HIV Develop Spike-Specific Lymph Node Germinal Center Responses following SARS-CoV-2 Vaccination. J Immunol 210, 947-958 (2023).
3. Turner, J. S. et al. Human germinal centres engage memory and naive B cells after influenza vaccination. Nature 586, 127-132 (2020).
4. Nutt, S. L., Hodgkin, P. D., Tarlinton, D. M. & Corcoran, L. M. The generation of antibody-secreting plasma cells. Nat Rev Immunol 15, 160-171 (2015).
5. MacLennan, I. C. et al. Extrafollicular antibody responses. Immunol Rev 194, 8-18 (2003).
6. Kurosaki, T., Kometani, K. & Ise, W. Memory B cells. Nature Reviews Immunology 15, 149-159 (2015).
7. MacLennan, I. C. et al. Extrafollicular antibody responses. Immunol Rev 194, 8-18 (2003).
8. Allen, C. D., Okada, T. & Cyster, J. G. Germinal-center organization and cellular dynamics. Immunity 27, 190-202 (2007).
9. De Silva, N. S. & Klein, U. Dynamics of B cells in germinal centres. Nat Rev Immunol 15, 137-148 (2015).
10. McHeyzer-Williams, M., Okitsu, S., Wang, N. & McHeyzer-Williams, L. Molecular programming of B cell memory. Nat Rev Immunol 12, 24-34 (2012).
11. Allen, C. D. et al. Germinal center dark and light zone organization is mediated by CXCR4 and CXCR5. Nat Immunol 5, 943-952 (2004).
12. Rodda, L. B., Bannard, O., Ludewig, B., Nagasawa, T. & Cyster, J. G. Phenotypic and Morphological Properties of Germinal Center Dark Zone Cxcl12-Expressing Reticular Cells. Journal of immunology 195, 4781-4791 (2015).
13. Scharer, C. D., Barwick, B. G., Guo, M., Bally, A. P. R. & Boss, J. M. Plasma cell differentiation is controlled by multiple cell division-coupled epigenetic programs. Nat Commun 9, 1698 (2018).
14. Shree, T. et al. Impaired Immune Health in Survivors of Diffuse Large B-Cell Lymphoma. J Clin Oncol 38, 1664-1675 (2020).
15. Beguelin, W. et al. EZH2 enables germinal centre formation through epigenetic silencing of CDKN1A and an Rb-E2F1 feedback loop. Nature communications 8, 877 (2017).
16. Kim, H. K. et al. Generation of human tonsil epithelial organoids as an ex vivo model for SARS-CoV-2 infection. Biomaterials 283, 121460 (2022).
17. Kastenschmidt, J. M. et al. Influenza vaccine format mediates distinct cellular and antibody responses in human immune organoids. Immunity 56, 1910-1926.e1917 (2023).
18. Yin, Q. et al. A TLR7-nanoparticle adjuvant promotes a broad immune response against heterologous strains of influenza and SARS-CoV-2. Nat Mater 22, 380-390 (2023).
19. Kastenschmidt, J. M. et al. Influenza vaccine format mediates distinct cellular and antibody responses in human immune organoids. Immunity 56, 1910-1926 e1917 (2023).
20. Wagar, L. E. et al. Modeling human adaptive immune responses with tonsil organoids. Nat Med 27, 125-135 (2021).
21. Victora, G. D. & Nussenzweig, M. C. Germinal Centers. Annu Rev Immunol 40, 413-442 (2022).
22. Allen, C. D. & Cyster, J. G. Follicular dendritic cell networks of primary follicles and germinal centers: phenotype and function. Semin Immunol 20, 14-25 (2008).
23. Singh, A. Eliciting B cell immunity against infectious diseases using nanovaccines. Nat Nanotechnol 16, 16-24 (2021).
24. Kwak, K. et al. Intrinsic properties of human germinal center B cells set antigen affinity thresholds. Sci Immunol 3 (2018).
25. Assen, F. P. et al. Multitier mechanics control stromal adaptations in the swelling lymph node. Nat Immunol 23, 1246-1255 (2022).
26. Horsnell, H. L. et al. Lymph node homeostasis and adaptation to immune challenge resolved by fibroblast network mechanics. Nat Immunol 23, 1169-1182 (2022).
27. Apoorva, F. N. U. et al. Lymph node stiffness-mimicking hydrogels regulate human B-cell lymphoma growth and cell surface receptor expression in a molecular subtype-specific manner. J Biomed Mater Res A 105, 1833-1844 (2017).
28. Huse, M. Mechanical forces in the immune system. Nature Reviews Immunology 17, 679-690 (2017).
29. Du, H. et al. Tuning immunity through tissue mechanotransduction. Nature Reviews Immunology 23, 174-188 (2023).
30. Purwada, A., Shah, S. B., Beguelin, W., Melnick, A. M. & Singh, A. Modular Immune Organoids with Integrin Ligand Specificity Differentially Regulate Ex vivo B Cell Activation. ACS Biomater Sci Eng 3, 214-225 (2017).
31. Phelps, E. A. et al. Maleimide cross-linked bioactive PEG hydrogel exhibits improved reaction kinetics and cross-linking for cell encapsulation and in situ delivery. Advanced materials 24, 64-70, 62 (2012).
32. Shah, S. B. et al. Combinatorial treatment rescues tumour-microenvironment-mediated attenuation of MALT1 inhibitors in B-cell lymphomas. Nat Mater 22, 511-523 (2023).
33. Luo, W., Weisel, F. & Shlomchik, M. J. B Cell Receptor and CD40 Signaling Are Rewired for Synergistic Induction of the c-Myc Transcription Factor in Germinal Center B Cells. Immunity 48, 313-326 e315 (2018).
34. Urashima, M., Chauhan, D., Uchiyama, H., Freeman, G. J. & Anderson, K. C. CD40 ligand triggered interleukin-6 secretion in multiple myeloma. Blood 85, 1903-1912 (1995).
35. Unger, P. A. et al. Minimalistic In vitro Culture to Drive Human Naive B Cell Differentiation into Antibody-Secreting Cells. Cells 10 (2021).
36. Burkle, A. et al. Overexpression of the CXCR5 chemokine receptor, and its ligand, CXCL13 in B-cell chronic lymphocytic leukemia. Blood 110, 3316-3325 (2007).
37. Tian, Y. F. et al. Integrin-specific hydrogels as adaptable tumor organoids for malignant B and T cells. Biomaterials 73, 110-119 (2015).
38. Lutolf, M. P. & Hubbell, J. A. Synthesis and physicochemical characterization of end-linked poly(ethylene glycol)-co-peptide hydrogels formed by Michael-type addition. Biomacromolecules 4, 713-722 (2003).
39. Darling, N. J., Hung, Y. S., Sharma, S. & Segura, T. Controlling the kinetics of thiol-maleimide Michael-type addition gelation kinetics for the generation of homogenous poly(ethylene glycol) hydrogels. Biomaterials 101, 199-206 (2016).
40. Baldwin, A. D. & Kiick, K. L. Tunable degradation of maleimide-thiol adducts in reducing environments. Bioconjug Chem 22, 1946-1953 (2011).
41. Schrock, D. C. et al. Pivotal role for alphaV integrins in sustained Tfh support of the germinal center response for long-lived plasma cell generation. Proc Natl Acad Sci USA 116, 4462-4470 (2019).
42. Clark, A. Y. et al. Integrin-specific hydrogels modulate transplanted human bone marrow-derived mesenchymal stem cell survival, engraftment, and reparative activities. Nature communications 11, 114 (2020).
43. Mittelbrunn, M. et al. VLA-4 integrin concentrates at the peripheral supramolecular activation complex of the immune synapse and drives T helper 1 responses. Proceedings of the National Academy of Sciences 101, 11058-11063 (2004).
44. Vazquez, M. I., Catalan-Dibene, J. & Zlotnik, A. B cells responses and cytokine production are regulated by their immune microenvironment. Cytokine 74, 318-326 (2015).
45. Purwada, A. et al. Ex vivo synthetic immune tissues with T cell signals for differentiating antigen-specific, high affinity germinal center B cells. Biomaterials 198, 27-36 (2019).
46. Martin, H. J., Lee, J. M., Walls, D. & Hayward, S. D. Manipulation of the toll-like receptor 7 signaling pathway by Epstein-Barr virus. J Virol 81, 9748-9758 (2007).
47. Koyama, S. et al. Plasmacytoid dendritic cells delineate immunogenicity of influenza vaccine subtypes. Sci Transl Med 2, 25ra24 (2010).
48. Jeisy-Scott, V. et al. TLR7 recognition is dispensable for influenza virus A infection but important for the induction of hemagglutinin-specific antibodies in response to the 2009 pandemic split vaccine in mice. J Virol 86, 10988-10998 (2012).
49. Bishop, G. A. et al. The immune response modifier resiquimod mimics CD40-induced B cell activation. Cell Immunol 208, 9-17 (2001).
50. Holbrook, B. C. et al. An R848 adjuvanted influenza vaccine promotes early activation of B cells in the draining lymph nodes of non-human primate neonates. Immunology 153, 357-367 (2018).
51. Holbrook, B. C. et al. A Novel R848-Conjugated Inactivated Influenza Virus Vaccine Is Efficacious and Safe in a Neonate Nonhuman Primate Model. J Immunol 197, 555-564 (2016).
52. Guthmiller, J. J., Graham, A. C., Zander, R. A., Pope, R. L. & Butler, N. S. Cutting Edge: IL-10 Is Essential for the Generation of Germinal Center B Cell Responses and Anti-Plasmodium Humoral Immunity. J Immunol 198, 617-622 (2017).
53. Auladell, M. et al. Distinguishing naive- from memory-derived human B cells during acute responses. Clin Transl Immunology 8, e01090 (2019).
54. Duan, M. et al. Understanding heterogeneity of human bone marrow plasma cell maturation and survival pathways by single-cell analyses. Cell Rep 42, 112682 (2023).
55. Becht, E. et al. Dimensionality reduction for visualizing single-cell data using UMAP. Nat Biotechnol (2018).
56. Barwick, B. G. et al. B cell activation and plasma cell differentiation are inhibited by de novo DNA methylation. Nat Commun 9, 1900 (2018).
57. Sanz, I. et al. Challenges and Opportunities for Consistent Classification of Human B Cell and Plasma Cell Populations. Front Immunol 10, 2458 (2019).
58. Mesin, L., Ersching, J. & Victora, G. D. Germinal Center B Cell Dynamics. Immunity 45, 471-482 (2016).
59. Beguelin, W. et al. EZH2 is required for germinal center formation and somatic EZH2 mutations promote lymphoid transformation. Cancer Cell 23, 677-692 (2013).
60. Guo, M. et al. EZH2 Represses the B Cell Transcriptional Program and Regulates Antibody-Secreting Cell Metabolism and Antibody Production. Journal of immunology 200, 1039-1052 (2018).
61. Duy, C. et al. BCL6 is critical for the development of a diverse primary B cell repertoire. J Exp Med 207, 1209-1221 (2010).
62. Jenks, S. A. et al. Distinct Effector B Cells Induced by Unregulated Toll-like Receptor 7 Contribute to Pathogenic Responses in Systemic Lupus Erythematosus. Immunity 49, 725-739 e726 (2018).
63. Tipton, C. M. et al. Diversity, cellular origin and autoreactivity of antibody-secreting cell population expansions in acute systemic lupus erythematosus. Nat Immunol 16, 755-765 (2015).
64. Silva-Cayetano, A. et al. Spatial dysregulation of T follicular helper cells impairs vaccine responses in aging. Nat Immunol 24, 1124-1137 (2023).
65. Shi, W. et al. Transcriptional profiling of mouse B cell terminal differentiation defines a signature for antibody-secreting plasma cells. Nat Immunol 16, 663-673 (2015).
66. Weiner, L. M., Murray, J. C. & Shuptrine, C. W. Antibody-based immunotherapy of cancer. Cell 148, 1081-1084 (2012).
67. Caspi, R. R. Immunotherapy of autoimmunity and cancer: the penalty for success. Nat Rev Immunol 8, 970-976 (2008).
68. Wennhold, K. et al. Using Antigen-Specific B Cells to Combine Antibody and T Cell-Based Cancer Immunotherapy. Cancer Immunol Res 5, 730-743 (2017).
69. Kim, S., Shah, S. B., Graney, P. L. & Singh, A. Multiscale engineering of immune cells and lymphoid organs. Nat Rev Mater 4, 355-378 (2019).
70. Hagan, T. et al. Transcriptional atlas of the human immune response to 13 vaccines reveals a common predictor of vaccine-induced antibody responses. Nat Immunol 23, 1788-1798 (2022).
71. Fourati, S. et al. Pan-vaccine analysis reveals innate immune endotypes predictive of antibody responses to vaccination. Nat Immunol 23, 1777-1787 (2022).
72. Moeller, T. D. et al. Profiling Germinal Center-like B Cell Responses to Conjugate Vaccines Using Synthetic Immune Organoids. ACS Cent Sci 9, 787-804 (2023).
73. Jonges, M. et al. Influenza virus inactivation for studies of antigenicity and phenotypic neuraminidase inhibitor resistance profiling. J Clin Microbiol 48, 928-940 (2010).
74. Han, G., Spitzer, M. H., Bendall, S. C., Fantl, W. J. & Nolan, G. P. Metal-isotope-tagged monoclonal antibodies for high-dimensional mass cytometry. Nat Protoc 13, 2121-2148 (2018).
75. Allam, M., Cai, S. & Coskun, A. F. Multiplex bioimaging of single-cell spatial profiles for precision cancer diagnostics and therapeutics. NPJ Precis Oncol 4, 11 (2020).
76. Allam, M. et al. Spatially visualized single-cell pathology of highly multiplexed protein profiles in health and disease. Commun Biol 4, 632 (2021).
77. Xu, W. et al. Cell stiffness is a biomarker of the metastatic potential of ovarian cancer cells. PLoS One 7, e46609 (2012).
78. Apoorva, F. et al. How Biophysical Forces Regulate Human B Cell Lymphomas. Cell Rep 23, 499-511 (2018).
79. Fontan, L. et al. Identification of MALT1 feedback mechanisms enables rational design of potent antilymphoma regimens for ABC-DLBCL. Blood 137, 788-800 (2021).
80. Purwada, A. et al. Ex vivo engineered immune organoids for controlled germinal center reactions. Biomaterials 63, 24-34 (2015).

1. An organ-on-chip system, comprising:
a fluidic substrate having a plurality of channels and a chamber, the chamber being configured to house a hydrogel embedded with a plurality of immune cells or placed thereon;
wherein the plurality of channels include at least one channel connected to the chamber to deliver a stimulating agent to the plurality of immune cells of the hydrogel;
wherein the hydrogel comprises a gradient of the stimulating agent; and
wherein different regions of the hydrogel each with different portions of the plurality of immune cells express different phenotypes relative to their placement within the gradient.

2. The organ-on-chip system of claim 1, wherein the hydrogel comprises polyethylene glycol (PEG), collagen, alginate, gelatin, fibronectin, fibrinogen, laminin, hyaluronic acid, extracellular matrix, Matrigel, or any combination thereof.

3-5. (canceled)

6. The organ-on-chip system of claim 1, wherein the hydrogel is functionalized with one or more peptides.

7. The organ-on-chip system of claim 6, wherein the one or more peptides comprise GYGGGP(GPP)5GFOGER(GPP)5GPC, where O=hydroxyproline, GRGDSPC, GREDVGC, or any combination thereof.

8. (canceled)

9. The organ-on-chip system of claim 1, wherein the stimulating agent comprises a chemokine, a cytokine, a small molecule, or any combination thereof.

10-11. (canceled)

12. The organ-on-chip system of claim 9, wherein the small molecule is an EZH2 inhibitor, a Bc16 inhibitor, a TLR7/8 agonist, or any combination thereof.

13. (canceled)

14. The organ-on-chip system of claim 1, wherein the hydrogel further comprises two opposing gradients of two different stimulating agents.

15. The organ-on-chip system of claim 1, further comprising a first inlet for a cell culture media and a second inlet for the stimulating agent.

16. The organ-on-chip system of claim 15, wherein each of the plurality of channels are in fluid communication with the first inlet and the second inlet; and
wherein each of the plurality of channels closer to the first inlet have a lower concentration of the stimulating agent and each of the plurality of channels closer to the second inlet have a higher concentration of the stimulating agent.

17-22. (canceled)

23. A method of stimulating a plurality of immune cells, the method comprising culturing a plurality of immune cells on the organ-on-chip system of claim 1.

24. The method of claim 23, wherein the plurality of immune cells comprise B cells and/or T cells, dendritic cells, and subtypes, monocytes, macrophages, natural killer cells, neutrophils, follicular dendritic cells, and/or stromal cells.

25. The method of claim 23, further comprising collecting the stimulated immune cells and using said stimulated immune cells for immunotherapy.

26. A cell produced by the method of claim 25.

27-28. (canceled)

29. A method of monitoring disease progression, the method comprising:
a) culturing a plurality of immune cells on the organ-on-chip system of claim 1; and
b) observing the plurality of immune cells over a period of time;
wherein the plurality of immune cells are diseased or abnormal.

30. (canceled)

31. The method of claim 29, wherein the plurality of immune cells comprise lymphoma cells or multiple myeloma cells;
wherein the plurality of immune cells are infected with tuberculosis, RSV, HIV, malaria, coronavirus, or influenza; or
wherein the plurality of immune cells are consistent with lupus, rheumatoid arthritis, Evan's syndrome, or Hyper X-IgM.

32. (canceled)

33. (canceled)

34. The method of claim 29, wherein the plurality of immune cells are healthy, and step a) further comprises exposing the plurality of immune cells to a pathogen.

35-36. (canceled)

37. A method of determining a response to a therapeutic intervention, the method comprising:
a) culturing a plurality of immune cells on the organ-on-chip system of claim 1; and
b) subjecting the plurality of immune cells to the therapeutic intervention.

38. The method of claim 37, wherein the plurality of immune cells are diseased or abnormal, and the therapeutic intervention is a therapeutic agent, chemotherapy, radiation, high-intensity focused ultrasound, or any combination thereof, or
wherein the plurality of immune cells are healthy, and the therapeutic intervention is a vaccination.

39-48. (canceled)

49. A method of generating antibodies, the method comprising:
a) culturing a plurality of immune cells on the organ-on-chip system of claim 1; and
b) collecting antibodies produced by the plurality of immune cells.

50. The method of claim 49, wherein step a) further comprises B cell clone development.

51-56. (canceled)
